(19) United States
(12) Patent Application Publication
(10) Pub. No.: US 2026/0283125 A1
(43) Pub. Date: Sep. 24, 2026

(54) METHOD FOR GENDER DETERMINATION OF CHICKEN EMBRYOS

(71) Applicant: EGGXYT LTD., Jerusalem (IL)

(72) Inventors: Daniel Yehuda OFFEN, Kfar Haroe (IL); Yehuda ELRAM, Kfar Adumim (IL); Eliran KADOSH, Jerusalem (IL); Malkiel COHEN, Lehavim (IL)

(21) Appl. No.: 19/469,190

(22) PCT Filed: Mar. 27, 2024
(86) PCT No.: PCT/IL2024/050320

§ 371 Date: Sep. 25, 2025

Related U.S. Application Data
(60) Provisional application No. 63/454,804, filed on Mar. 27, 2023.

(51) Int. Cl. A01K 67/0278; C12N 9/22; C12N 15/11; C12N 15/90

(52) U.S. Cl. CPC A01K 67/0278; C12N 9/226; C12N 15/111; C12N 15/902; A01K 2217/072; A01K 2227/30; A01K 2267/0393; C12N 2310/10

(57) ABSTRACT

The present invention provides a transgenic female chicken, including a recombinant Z gender chromosome including a nucleic acid sequence of an exogenous reporter gene encoding a red fluorescent protein (RFP), in at least one location of the Z gender chromosome. Further provided is a method of gender determination of a chicken embryo in an unhatched egg.

### REFERENCE TO AN ELECTRONIC SEQUENCE LISTING

The contents of the electronic sequence listing (EGG-P-010-PCT.xml; size: 59,207 bytes; and date of creation: Mar. 21, 2024) is herein incorporated by reference in its entirety.

### CROSS-REFERENCE TO RELATED APPLICATION

This application claims the benefit of priory of U.S. Provisional Patent Application No. 63/454,804, titled “METHOD FOR GENDER DETERMINATION OF CHICKEN EMBRYOS”, filed 27 Mar. 2023, the contents of which are incorporated herein by reference in their entirety.

### FIELD OF THE INVENTION

The present invention relates to a transgenic chicken and methods of using same, such as for gender determination of a chicken embryo in an unhatched egg.

### BACKGROUND OF THE INVENTION

The food industry uses “broiler” chickens for chicken meat production. These chickens are known to reach slaughter weight between four and six weeks of age. On the other hand, egg-laying hens are mainly bred to put all their energy toward laying and thus rarely reach a sufficient weight for meat production. The males from the leaner breeds used in egg production cost more to feed and house than they would ever sell for as meat, so they are economically less favorable to the industry. This is why male chickens are culled by billions on a daily basis via suffocation or grinding, yielding a financial and ethical worldwide problem.

Chick sexing is the method of distinguishing the sex of chickens and other hatchlings, usually by a trained person called a chick sexer or chicken sexer. Several methods are used to determine the sex of at least one day-old chick, including vent sexing which is universal for all breeds, but requires the work of an expert, as well feather sexing, and color sexing that rely on a sex-linked slow-feathering gene, and on a sex-linked slow-feathering gene, respectively. However, there is still a great need for in-ovo sexing of an unhatched egg.

An automated system to determine the sex of a 14-day in-ovo developing chick has been previously described. This system is specifically based on the substantial difference in the feather color of males and females, that could be observed by candling a 14-days old egg. In this system, the egg is candled with a halogen lamp. Even though no negative effects were observed in the developing chicks, halogen lamps are known to produce significant amounts of heat that might have a negative impact on the embryo if the candling time is too long.

There is still a great need for new, robust, cost-effective, accurate methods for in-ovo sexing of embryos in unhatched eggs, specifically, such that can be generally applied to any chicken strain, and even more so, applicable in very early stages of embryonic development.

### SUMMARY OF THE INVENTION

According to one aspect, there is provided a transgenic female chicken comprising a recombinant Z gender chromosome comprising a nucleic acid sequence of an exogenous reporter gene encoding a red fluorescent protein (RFP), in at least one location of a Z gender chromosome of a female chicken, wherein the at least one location is listed under Table 1.

In some embodiments, the at least one location of a Z gender chromosome, as disclosed throughout the application, inclusive of Table 1, is in accordance with or based on the UCSC Genome Browser assembly ID: galGal6.

According to another aspect, there is provided a cell obtained or derived from the transgenic female chicken disclosed herein.

According to another aspect, there is provided a method of gender determination of a chicken embryo in an unhatched egg comprising the embryo within a structurally integral shell, the method comprising: (a) obtaining at least one unhatched egg comprising an embryo within a structurally integral shell, from the transgenic female chicken disclosed herein; and (b) determining whether a red fluorescent signal is detected in the embryo residing in the unhatched egg, wherein detection of the red fluorescent signal indicates the expression of the RFP in the embryo within the structurally integral shell of the unhatched egg, and thus indicates the presence of the recombinant Z chromosome in the embryo, thereby determining that the chicken embryo in the unhatched egg is a male embryo.

According to another aspect, there is provided a method for producing a chicken comprising a recombinant Z gender chromosome, the method comprising: (a) obtaining at least one transgenic PGC comprising a recombinant Z chromosome comprising a nucleic acid sequence of an exogenous reporter gene encoding an RFP in at least one location as listed under Table 1 in Z gender chromosome of a chicken; and, (b) transplanting the transgenic PGC into a receptive chicken embryo, thereby producing a chicken comprising the recombinant Z gender chromosome.

According to another aspect, there is provided a kit comprising at least one first nucleic acid molecule encoding a guide RNA (gRNA), comprising the nucleotide sequence as set forth in any one of SEQ ID Nos: 1-3, and 18-20.

In some embodiments, the at least one location is selected from the group consisting of: site 3, site 4, site 5, site 7, site 8, site 13, site 14, site 15, and any combination thereof.

In some embodiments, the at least one location is listed under Table 3.

In some embodiments, the at least one location is selected from the group consisting of: site 4a, site 5a, site 7b, site 14a, and any combination thereof.

In some embodiments, the at least one location is selected from the group consisting of: site 4a, site 13a, site 14a, and any combination thereof.

In some embodiments, the RFP is characterized by an excitation wavelength of 500-650 nm and an emission wavelength of 550-650 nm.

In some embodiments, the recombinant Z gender chromosome comprises the nucleotide sequence set forth in any one of SEQ ID Nos: 4-6.

In some embodiments, the cell is a primordial germ cell (PGC).

In some embodiments, detection of no red fluorescent signal in the embryo residing in the unhatched egg indicates that RFP is not expressed in the embryo, and thus indicates the absence of the recombinant Z chromosome in the embryo, thereby determining that the chicken embryo in the unhatched egg is a female embryo.

In some embodiments, the method further comprises a step comprising subjecting the unhatched egg comprising the embryo within a structurally integral shell to a light source.

In some embodiments, the method further comprises a step proceeding step (b) comprising isolating at least one female embryo of the transgenic female chicken.

In some embodiments, the kit further comprises at least one second nucleic acid molecule encoding any one of: a clustered regularly interspaced short palindromic repeat (CRISPR)-associated protein 9 (Cas9), an RFP, and both.

In some embodiments, the kit further comprises instructions for integrating the at least second nucleic acid molecule encoding RFP into at least one location of Z gender chromosome of a female chicken, wherein the at least one location is listed under Table 1.

In some embodiments, the method further comprises a step preceding step (a), comprising integrating the nucleic acid sequence of an exogenous reporter gene encoding an RFP into at least one location as listed under Table 1 in Z gender chromosome of a PGC, thereby obtaining the transgenic PGC.

In some embodiments, the nucleic acid sequence of an exogenous reporter gene encoding an RFP is integrated into the location of the Z gender chromosome using CRISPR type II system, comprising Cas9 protein and a gRNA.

In some embodiments, the gRNA comprises the nucleotide sequence set forth in any one of SEQ ID Nos: 1-3, and 18-20.

In some embodiments, the nucleic acid sequence of an exogenous reporter gene is integrated into site 4a, site 5a, or site 14a, using Cas9 protein and a gRNA comprising the nucleotide sequence as set forth in SEQ ID NO: 1, SEQ ID NO: 20, or SEQ ID NO: 3, respectively.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

In addition to the exemplary aspects and embodiments described above, further aspects and embodiments will become apparent by reference to the study of the following detailed description.

### BRIEF DESCRIPTION OF THE DRAWINGS

FIG. 1 includes an image of gel electrophoresis demonstrating cleavage products by a T7EI nuclease of double stranded DNA (dsDNA) molecules from DF1 cells. Genomic DNA was edited by CRISPR-associated protein 9 (Cas9) and a guide RNA molecule (gRNA) selected from: gRNA6.1, gRNA7.2, and gRNA9.1, and PCR amplified prior cleavage by T7EI nuclease. +, with gRNA molecule; −, without gRNA molecule; m, marker.

FIG. 2 includes a bar graph of tracking of indels by decomposition (TIDE) analyses demonstrating the frequency of DNA insertions and deletions (indels) in DF1 genomic DNA treated with Cas9/gRNA ribonucleoprotein (RNP) complexes. Indels were observed with greater frequency in genomic DNA targeted by either gRNA6.1, gRNA7.2, or gRNA9.1, compared to other gRNAs molecules.

FIG. 3 includes photographs of agarose gel electrophoresis of T7 assay evaluating the efficiency of designed guide RNAs (gRNAs). For sites 4a and 14a, the inventors utilized previously validated gRNAs (gRNA 6.1 and 9.1, respectively), known for their efficacy in inducing double strand break (DSB) at the targeted genomic loci. For safe harbor loci (SHL) 7b, 8a, and 13a, the inventors designed and tested 2, 4, and 4 gRNAs, respectively. The symbols “−” and “+” indicate samples before and after treatment with T7 endonuclease, respectively. The non-transfected control depicts PCR products obtained using equivalent T7 primers without RNP electroporation. In the assay, gRNAs 6.1, 6.1 #2, and 7.2 were employed as positive controls, representing RNP complexes with confirmed cleavage capability, ensuring the fidelity of the T7 assay. Additionally, “NTC” denotes the no-template control, serving to demonstrate the absence of contaminations in the PCR assay.

FIGS. 4A-4F include fluorescent micrographs showing genomic integration of transgene in two SHL. DsRed (4A and 4D) and EGFP (4B and 4E) fluorescence observed 2-3 days post-transfection of Lohmann-LSL PGC targeted at two different genomic SHL, representing expression from DsRed HDR and CRISPR/Cas9 plasmids, respectively. The merge field (4C and 4F) demonstrates colocalization of red and green fluorescence in some of the cells.

FIGS. 5A-5B include fluorescent micrographs showing stable primordial germ cell (PGC) cultures expressing DsRed targeted at two SHL: 4a (5A) and 13a (5B).

FIGS. 6A-6B include photographs of agarose gel electrophoresis of PCR assays confirming the accurate integration of the DsRed coding gene at SHL 4a (6A) and 13a (6B) are depicted, with the expected product sizes. For SHL 13a, two sets of primers (#1 and #2) were employed for each homology arm.

FIGS. 7A-7C includes fluorescent micrographs showing in-ovo expression of DsRed in PGC harboring the DsRed encoding gene incorporated in a SHL in the genome. Non-fertile eggs were injected with: control non-edited PGC (7B) or edited PGC harboring DsRed encoding gene in a SHL in the genome in an amount equivalent to the cell number in embryonic day-3 (7C), and subsequently underwent scanning using an electro-optical scanner. A distinct fluorescent focus was only detectable in the egg injected with DsRed+ cells, as indicated by the arrow (7C). (7A) A control non-treated egg.

### DETAILED DESCRIPTION OF THE INVENTION

The present invention, in some embodiments, provides a transgenic female chicken comprising a recombinant Z gender chromosome comprising a nucleic acid sequence of an exogenous reporter gene encoding a red fluorescent protein (RFP). The present invention is further directed to a method of gender determination of a chicken embryo in an unhatched egg comprising the embryo within a structurally integral shell. Also provided herein are a kit comprising at least one nucleic acid molecule encoding a guide RNA (gRNA), comprising a nucleotide sequence as set forth in SEQ ID Nos: 1-3, and 18-20, and a method for producing a chicken comprising a recombinant Z gender chromosome.

### Transgenic Chicken, Cell Thereof, and Method for Producing Thereof.

According to another aspect, there is provided a transgenic female chicken comprising a recombinant Z gender chromosome comprising a nucleic acid sequence of an exogenous reporter gene encoding a red fluorescent protein (RFP), in at least one location of the Z gender chromosome, wherein the at least one location is listed under Table 1.

According to another aspect, there is provided a transgenic chicken cell comprising a recombinant Z gender chromosome comprising a nucleic acid sequence of an exogenous reporter gene encoding a red fluorescent protein (RFP), in at least one location of the Z gender chromosome, wherein the at least one location is listed under Table 1.

In some embodiments, the transgenic chicken cell is a stable cell. In some embodiments, the transgenic chicken cell is genomically stable.

In some embodiments, the stability, genomic stability, fitness, survival, viability, or any combination thereof, of the transgenic cell of the invention, is essentially similar to a control. In some embodiments, a control is or comprises a control chicken cell.

In some embodiments, a control chicken cell comprises a wildtype chicken cell, a cell derived from or of a genetic background or reference of the transgenic chicken cell, or both.

In some embodiments, “essentially similar” refers to being at least 80%, 90%, 95%, 97%, or 99% similar to a control cell as described herein, or any value and range therebetween. In some embodiments, essentially similar refers to being 80-100%, 85-100%, 90-100%, 95-100%, or 97-100% similar to a control cell as described herein. Each possibility represents a separate embodiment of the invention.

In some embodiments, the transgenic chicken cell is characterized by expression of RFP being comparable to a control, proliferation rate being comparable to a control, or both.

In some embodiments, the transgenic chicken cell is characterized by stable expression of RFP, stable proliferation rate, or both. In some embodiments, stable is comparable to a control.

In some embodiments, stable is stable over or across time. In some embodiments, stable is for a period of time. In some embodiments, stable is for at least 1 passage, 2 passages, 4 passages, 5 passages, or any value and range therebetween. Each possibility represents a separate embodiment of the invention.

In some embodiments, stable is for at least 1 week, 2 weeks, 3 weeks, 4 weeks, 2 months, 4 months, 6 months, or any value and range therebetween. In some embodiments, stable is for 1 to 3 weeks, 2 to 6 weeks, 3 to 7 weeks, 1 to 3 months, 2 to 6 months, or 4 to 6 months. Each possibility represents a separate embodiment of the invention.

The terms, “comparable” and “essentially similar” are used herein interchangeably.

In some embodiments, essentially similar comprises or is 100% similar, e.g., ‘identical’.

As used herein, the terms “stable” or “genomically stable” refer to the cell not losing fitness, survival, viability, activity, or the like, due to the presence of the nucleic acid sequence of the exogenous reporter gene encoding a red fluorescent protein (RFP), in at least one location of the Z gender chromosome of the cell, wherein the at least one location is listed under Table 1.

In some embodiments, the transgenic chicken cell of the invention is characterized by a consistently stable RFP expression. In some embodiments, the transgenic chicken cell of the invention is characterized by sustained proliferation. In some embodiments, the transgenic chicken cell of the invention is characterized by a consistently stable RFP expression and sustained proliferation.

Methods for determining RFP expression over time (e.g., consistent stable RFP expression) as well as cell proliferation over time (e.g., sustained proliferation) are common and would be apparent to one of ordinary skill in the art, such as, exemplified herein.

The term “transgenic chicken”, as used herein, refers to a chicken (Gallus gallus) which is genetically modified such that the genome of the chicken comprises an exogenous DNA sequence incorporated therein. In some embodiments, the exogenous DNA sequence is integrated into a gender chromosome of a germ cell of the transgenic chicken. As a result of such integration, the exogenous sequence may be transmitted through germ cells to the offspring of a transgenic chicken. The transgenic chicken, including its progeny, also has the exogenous reporter gene integrated into the gender chromosomes of somatic cells. As used herein, the terms: “location”, “locus”, and “genomic site” are interchangeable and refer to a site within a chromosome, in which the exogenous reporter gene can be integrated. A genomic site may refer to a location between two consecutive nucleotides in the genomic DNA, as well as to a range of consecutive nucleotides comprising up to 6,000,000 nucleotides. The term “Z gender chromosome”, as used herein refers to the “male” sex chromosome in chickens. As used herein, the term “recombinant Z gender chromosome” refers to a Z gender chromosome of a chicken, that has been genetically modified to include a nucleic acid of an exogenous reporter gene encoding RFP. In some embodiments, genetic modification performed by a gene editing tool or a system.

In some embodiments, the at least one location is selected from: site 1, comprising nucleotide number 1,550,000 to nucleotide number 1,730,000 of a chicken Z gender chromosome, site 2, comprising nucleotide number 4,075,000 to nucleotide number 6,150,000 of a chicken Z chromosome, site 3, comprising nucleotide number 17,100,000 to nucleotide number 17,450,000, site 4, comprising nucleotide number 17,550,000 to nucleotide number 18,050,000, site 5, comprising nucleotide number 19,500,000 to nucleotide number 20,000,000, site 6, comprising nucleotide number 26,000,000 to nucleotide number 26,400,000, site 7, comprising nucleotide number 30,000,000 to nucleotide number 30,800,000, site 8, comprising nucleotide number 36,000,000 to nucleotide number 36,600,000, site 9, comprising nucleotide number 38,500,000 to nucleotide number 39,200,000, site 10, comprising nucleotide number 48,300,000 to nucleotide number 50,000,000, site 11, comprising nucleotide number 50,900,000 to nucleotide number 51,400,000, site 12, comprising nucleotide number 51,425,000 to nucleotide number 51,625,000, site 13, comprising nucleotide number 58,500,000 to nucleotide number 59,700,000, site 14, comprising nucleotide number 61,400,000 to nucleotide number 62,400,000, site 15, comprising nucleotide number 63,150,000 to nucleotide number 63,550,000, site 16, comprising nucleotide number 64,350,000 to nucleotide number 65,250,000, site 17, comprising nucleotide number 69,700,000 to nucleotide number 70,400,000, site 18, comprising nucleotide number 73,500,000 to nucleotide number 78,800,000, or any combination thereof.

In some embodiments, at least one location comprises 1, 2, 3, 4, 5, 6, 7, 8, 9, 10, 11, 12, 13, 14, 15, 16, 17, or 18 locations selected from site Nos. 1-18, as disclosed herein, or any combination thereof. Each possibility represents a separate embodiment of the invention. In some embodiments, at least one location is one location selected from site Nos. 1-18, as disclosed herein.

In some embodiments, at least one location is selected from: site 3, site 4, site 5, site 7, site 8, site 13, site 14, site 15, or any combination thereof. In some embodiments, at least one location comprises 1, 2, 3, 4, 5, 6, 7, or 8, locations selected from site Nos. 3, 4, 5, 7, 8, 13, 14 and 15, as disclosed herein, or any combination thereof. Each possibility represents a separate embodiment of the invention. In some embodiments, at least one location is one location selected from site Nos. 3, 4, 5, 7, 8, 13, 14 and 15, as disclosed herein.

In some embodiments, at least one location is listed under Table 3. In some embodiments, site 3 comprises nucleotide number 17,141,000 to nucleotide number 17,406,000 (site 3a) of a chicken Z gender chromosome, site 4 comprises nucleotide number 17,735,000 to nucleotide number 18,020,000 (site 4a), site 5 comprises nucleotide number 19,560,000 to nucleotide number 19,995,000 (site 5a), site 7 comprises nucleotide number 30,060,000 to nucleotide number 30,270,000 (site 7a), or nucleotide number 30,465,000 to nucleotide number 30,750,000 (site 7b), site 8 comprises nucleotide number 36,215,000 to nucleotide number 36,545,000 (site 8a), site 13 comprises nucleotide number 59,100,000 to nucleotide number 59,430,000 (site 13a), site 14 comprises nucleotide number 61,530,000 to nucleotide number 61,865,000 (site 14a), or between nucleotide number 61,875,000 and nucleotide number 62,315,000 (site 14b), and site 15 comprises nucleotide number 63,210,000 to nucleotide number 63,440,000 (site 15a).

In some embodiments, at least one location comprises 1, 2, 3, 4, 5, 6, 7, 8, 9, or 10, locations selected from site Nos. 3a, 4a, 5a, 7a, 7b, 8a, 13a, 14a, 14b and 15a, as disclosed herein, or any combination thereof. Each possibility represents a separate embodiment of the invention. In some embodiments, at least one location is one location selected from site Nos. 3a, 4a, 5a, 7a, 7b, 8a, 13a, 14a, 14b and 15a, as disclosed herein.

In some embodiments, at least one location is selected from: site 4a, site 5a, site 7b, site 13a, site 14a, or any combination thereof. In some embodiments, at least one location comprises y 1, 2, 3, or 4, locations selected from site Nos. 4a, 5a, 7b, 13a, and 14a, as disclosed herein, or any combination thereof. Each possibility represents a separate embodiment of the invention. In some embodiments, at least one location is one location selected from site Nos. 4a, 5a, 7b, 13a, and 14a, as disclosed herein.

In some embodiments, at least one location is selected from: site 4a, site 5a, site 13a, site 14a, or any combination thereof. In some embodiments, at least one location comprises 1, 2, or 3, locations selected from site Nos. 4a, 5a, 13a, and 14a, as disclosed herein, or any combination thereof. Each possibility represents a separate embodiment of the invention. In some embodiments, at least one location is one location selected from site Nos. 4a, 5a, 13a, and 14a, as disclosed herein.

As used herein, the term “reporter gene” encompasses a gene which encodes a polypeptide, whose expression can be detected in a variety of known assays and wherein the level of the detected signal indicates the presence of the reported.

As used herein, the term “red fluorescent protein” or “RFP”, refers to a fluorophore that emits orange, red, and far-red fluorescence that has been isolated from anthozoans or anemones, or any variant thereof. In some embodiments, RFP comprises DsRed protein. In some embodiments, DsRed protein is isolated from Discosoma striata. In some embodiments, RFP comprises Kaede protein, isolated from Trachyphyllia geoffroyi. In some embodiments, RFP comprises an RFP variant. In some embodiments, the RFP variant disclosed herein fluoresce orange, red, far-red, or any combination thereof. In some embodiments, the RFP variant comprises a monomeric variant. In some embodiments, the RFP variant comprises reduced time between protein synthesis and expression of fluorescence (e.g., maturation time), as compared to DsRed. Variants of RFP are known in the art. Non limiting examples include mFruits (mCherry, mOrange, mRaspberry), mKO, TagRFP, mKate, mRuby, FusionRed, mScarlet and DsRed-Express.

In some embodiments, RFP comprises or is DsRed-Express

In some embodiments, an RFP or an analog thereof is characterized by or has excitation at a wavelength of 500-650 nm. In some embodiments, an RFP or analog thereof is characterized by or has excitation at a wavelength of 510-650, 520-650, 530-650, 540-650, 550-650, 500-600, 510-600, 520-600, 530-600, 540-600, 550-600, 500-590, 510-590, 520-590, 530-590, 540-590, 550-590, 500-580, 510-580, 520-580, 530-580, 540-580, 550-580, 500-570, 510-570, 520-570, 530-570, 540-570, 550-570, 500-560, 510-560, 520-560, 530-560, 540-560, or 550-560 nm. Each possibility represents a separate embodiment of the invention.

In some embodiments, an RFP or an analog thereof is characterized by or has emission at a wavelength of 550-650 nm. In some embodiments, an RFP or analog thereof is characterized by or has excitation at a wavelength of 500-650, 510-650, 520-650, 530-650, 540-650, 550-650, 560-650, 570-650, 580-650, 500-640, 510-640, 520-640, 530-640, 540-640, 550-640, 560-640, 570-640, 580-640, 500-630, 510-630, 520-630, 530-630, 540-630, 550-630, 560-630, 570-630, 580-630, 500-620, 510-620, 520-620, 530-620, 540-620, 550-620, 560-620, 570-620, 580-620, 500-610, 510-610, 520-610, 530-610, 540-610, 550-610, 560-610, 570-610, 580-610, 500-600, 510-600, 520-600, 530-600, 540-600, 550-600, 560-600, 570-600, 580-600, 500-590, 510-590, 520-590, 530-590, 540-590, 550-590, 560-590, 570-590, or 580-590 nm. Each possibility represents a separate embodiment of the invention.

Further examples for RFP analogs are disclosed in WO2017094015A1, which is incorporated herein by reference in its entirety.

In one embodiment, the maximum excitation of RFP is about 554 nm. In some embodiment, the maximum emission of RFP is about 586 nm.

The term “about” as used herein indicates values that may deviate by up to: 1%, 5%, 10%, 15%, or 20% higher or lower than the value referred to, the deviation range including integer values, and, if applicable, non-integer values as well, constituting a continuous range. As used herein the term “about” refers to +10%.

In some embodiments, the RFP disclosed herein comprises the amino acid sequence: MASSEDVIKEFMRFKVRMEGSVNGHEFEIEGEGEGRPYEGTQTAKLKVTKGGPLPFA WDILSPQFQYGSKVYVKHPADIPDYKKLSFPEGFKWERVMNFEDGGVVTVTQDSSLQ DGSFIYKVKFIGVNFPSDGPVMQKKTMGWEASTERLYPRDGVLKGEIHKALKLKDGG HYLVEFKSIYMAKKPVQLPGYYYVDSKLDITSHNEDYTIVEQYERAEGRHHLFL (SEQ ID NO: 7), or an analog or variant thereof.

The terms “variant” and “analog” are herein used interchangeably. An analog of RFP polypeptide includes any polypeptide, that is similar, but not identical, to a polypeptide comprising the amino acid sequence set forth in SEQ ID NO: 7, as long as it has or maintains at least 70%, 80%, 90% or 95%, 99%, or any value and range therebetween, of the maximum excitation and/or the maximum emission of RFP as disclosed herein. Each possibility represents a separate embodiment of the invention. In some embodiments, an analog of RFP polypeptide maintains 70%-100%, 80%-100%, 90%-100%, 95%-100%, or 99%-100% of the maximum excitation and/or the maximum emission of RFP as disclosed herein. Each possibility represents a separate embodiment of the invention. It should be understood that an analog of RFP refers to any fluorescent protein characterized by being capable of excitation at wavelength of 500-650 nm and of emission at wavelength of 550-650 nm.

In some embodiments, the reporter gene encoding RFP comprises an optimized codon sequence for expression in a chicken cell. As used herein, the term “an optimized codon sequence” describes a sequence that encodes identical amino acids to those encoded by a non-optimized codon sequence (synonymous codon), however, at least one of: translation rate of the codon optimized sequence, protein product amount, duration of protein structure stability, or any combination thereof, is increased, compared to the non-optimized codon. An ordinary skill in the art will know how to optimize a codon sequence for its expression in the desired cell, using a codon optimization gene engineering tool, comprising, but not limited to, algorithms that analyze codon optimization based on the codon frequencies in the desired cell/specie. In some embodiments, increased one of: translation rate, protein product amount, and duration of structure stability, is by at least by 30%.

In some embodiments, the reporter gene encoding RFP comprises the nucleic acid sequence:

(SEQ ID NO: 8)
ATGGCCTCCTCCGAGGACGTCATCAAGGAGTTCATGCGCTTCAAGGTGC
GCATGGAGGGCTCCGTGAACGGCCACGAGTTCGAGATCGAGGGCGAGGG
CGAGGGCCGCCCCTACGAGGGCACCCAGACCGCCAAGCTGAAGGTGACC
AAGGGCGGCCCCCTGCCCTTCGCCTGGGACATCCTGTCCCCCCAGTTCC
AGTACGGCTCCAAGGTGTACGTGAAGCACCCCGCCGACATCCCCGACTA
CAAGAAGCTGTCCTTCCCCGAGGGCTTCAAGTGGGAGCGCGTGATGAAC
TTCGAGGACGGCGGCGTGGTGACCGTGACCCAGGACTCCTCCCTGCAGG
ACGGCTCCTTCATCTACAAGGTGAAGTTCATCGGCGTGAACTTCCCCTC
CGACGGCCCCGTAATGCAGAAGAAGACTATGGGCTGGGAGGCCTCCACC
GAGCGCCTGTACCCCCGCGACGGCGTGCTGAAGGGCGAGATCCACAAGG
CCCTGAAGCTGAAGGACGGCGGCCACTACCTGGTGGAGTTCAAGTCCAT
CTACATGGCCAAGAAGCCCGTGCAGCTGCCCGGCTACTACTACGTGGAC
TCCAAGCTGGACATCACCTCCCACAACGAGGACTACACCATCGTGGAGC
AGTACGAGCGCGCCGAGGGCCGCCACCACCTGTTCCTGTAG.

In some embodiments, the reporter gene encoding RFP comprises the nucleic acid sequence as set forth in SEQ ID NO: 8, or an analog thereof, having at least 50%, 60%, 70%, 80%, 90%, 95%, 99% identity or homology thereto, or any value and range therebetween. Each possibility represents a separate embodiment of the invention. In some embodiments, an analog of RFP, comprising 50-100%, 60-100%, 70%-100%, 80%-100%, 90%-100%, 95%-100%, 99%-100% identity or homology to a nucleic acid sequence as set forth in SEQ ID NO: 8. Each possibility represents a separate embodiment of the invention.

In some embodiments, an analog of RFP comprises a protein translated by a nucleic acid molecule comprising a nucleotide sequence having at least 50%, 60%, 70%, 80%, 90%, 95%, 99% identity or homology to a nucleotide sequence as set forth in SEQ ID NO: 8, or any value and range therebetween. Each possibility represents a separate embodiment of the invention. In some embodiments, an analog of RFP comprises a protein translated by a nucleic acid molecule comprising a nucleotide sequence having 50%-100%, 60%-100%, 70%-100%, 80%-100%, 90%-100%, 95%-100%, 99%-100% identity or homology to a nucleotide sequence as set forth in SEQ ID NO: 8. Each possibility represents a separate embodiment of the invention.

In some embodiments, the reporter gene encoding RFP is integrated into the genomic site in the Z chromosome using a programmable engineered nuclease (PEN).

The term “programmable engineered nuclease (PEN)” as used herein, refers to a synthetic enzyme that cuts specific DNA sequences, derived from natural occurring nucleases involved in DNA repair of double strand DNA lesions, and enables direct genome editing.

In some embodiments, PEN used by the methods of the invention may be any one of a clustered regularly interspaced short palindromic repeat (CRISPR) class I or class II system.

As used herein, “CRISPR” or “CRISP arrays” also known as SPIDRs (Spacer Interspersed Direct Repeats) constitute a family of recently described DNA loci that are usually specific to a particular bacterial species. The CRISPR array is a distinct class of interspersed short sequence repeats (SSRs) that were first recognized in E. coli. In subsequent years, similar CRISPR arrays were found in Mycobacterium tuberculosis, Haloferax mediterranei, Methanocaldococcus jannaschii, Thermotoga maritima and other bacteria and archaea. It should be understood that the invention contemplates the use of any of the known CRISPR systems, particularly and of the CRISPR systems disclosed herein. The CRISPR-Cas system, targets DNA molecules based on short homologous DNA sequences, called spacers that exist between repeats. These spacers guide CRISPR-associated (Cas) proteins to matching (and/or complementary) sequences within the foreign DNA, called proto-spacers, which are subsequently cleaved. The spacers can be rationally designed to target any DNA sequence. Moreover, this recognition element may be designed separately to recognize and target any desired target. With respect to CRISPR systems, as will be recognized by those skilled in the art, the structure of a naturally occurring CRISPR locus includes a number of short repeating sequences generally referred to as “repeats”. The repeats occur in clusters and are usually regularly spaced by unique intervening sequences referred to as “spacers.” Typically, CRISPR repeats vary from about 24 to 47 base pair (bp) in length and are partially palindromic. The spacers are located between two repeats and typically each spacer has unique sequences that are from about 20 or less to 72 or more bp in length. In some embodiments the CRISPR spacers used in the sequence encoding at least one gRNA of the methods and kits of the invention comprise between 10 to 75 nucleotides (nt) each. In some embodiments, the gRNA comprises at least: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or any vale and range therebetween. Each possibility represents a separate embodiment of the invention. In some embodiments, the gRNA comprises 70 to 150 nt. In some embodiments, the spacers comprise 20 to 35 nucleotides. In addition to at least one repeat and at least one spacer, a CRISPR locus also includes a leader sequence and optionally, a sequence encoding at least one tracrRNA. The leader sequence typically is an AT-rich sequence of up to 550 bp directly adjoining the 5′ end of the first repeat.

In some embodiments, the PEN used by the methods of the invention may be a CRISPR Class 2 system. In yet some further particular embodiments, such class 2 system may be a CRISPR type II system. In some embodiments, PEN comprises CRISPR type II system.

The term “CRISPR type II” system refers to a bacterial immune system that has been modified for genome engineering. It should be appreciated however that other genome engineering approaches, like zinc finger nucleases (ZFNs) or transcription-activator-like effector nucleases (TALENs) that relay upon the use of customizable DNA-binding protein nucleases that require design and generation of specific nuclease-pair for every genomic target may be also applicable herein. CRISPR-Cas systems fall into two classes. Class 1 systems use a complex of multiple Cas proteins to degrade foreign nucleic acids. Class 2 systems use a single large Cas protein for the same purpose. More specifically, class 1 may be divided into types I, III, and IV and class 2 may be divided into types II, V, and VI.

The type II CRISPR-Cas systems include the ‘HNH’-type system (Streptococcus-like; also known as the Nmeni subtype, for Neisseria meningitidis serogroup A str. Z2491, or CASS4), in which Cas9 is sufficient for generating crRNA and cleaving the target DNA, in addition to the ubiquitous Cas1 and Cas2. Cas9 contains at least two nuclease domains, a RuvC-like nuclease domain near the amino terminus and the HNH (or McrA-like) nuclease domain in the middle of the protein, but the function of these domains remains to be elucidated. However, as the HNH nuclease domain is abundant in restriction enzymes and possesses endonuclease activity responsible for target cleavage.

Type II systems cleave the pre-crRNA through an unusual mechanism that involves duplex formation between a tracrRNA and part of the repeat in the pre-crRNA; the first cleavage in the pre-crRNA processing pathway subsequently occurs in this repeat region. Still further, it should be noted that type II system comprise at least one of Cas9, Cas1, Cas2 csn2, and Cas4 genes. It should be appreciated that any type II CRISPR-Cas systems may be applicable in the present invention, specifically, any one of type II-A or B.

In some embodiments, the at least one Cas gene used in the methods and kits of the invention may be at least one Cas gene of type II CRISPR system (either typeII-A or typeII-B). In some embodiments, at least one Cas gene of type II CRISPR system used by the methods and kits of the invention is the Cas9 gene. It should be appreciated that such system may further comprise at least one of Cas1, Cas2, csn2 and Cas4 genes.

In some embodiments, a Cas protein consists or comprise a Cas9 protein. Double-stranded DNA (dsDNA) cleavage by Cas9 is a hallmark of “type II CRISPR-Cas” immune systems. The CRISPR-associated protein Cas9 is an RNA-guided DNA endonuclease that uses RNA: DNA complementarity to identify target sites for sequence-specific double stranded DNA (dsDNA) cleavage, creating the double strand brakes (DSBs) required for the HDR that results in the integration of the reporter gene into the specific target sequence, for example, a specific target within the avian gender chromosome Z. The targeted DNA sequences are specified by the CRISPR array, which is a series of about 30 to 40 bp spacers separated by short palindromic repeats. The array is transcribed as a pre-crRNA and is processed into shorter crRNAs that associate with the Cas protein complex to target complementary DNA sequences known as proto-spacers. These proto-spacer targets must also have an additional neighboring sequence known as a proto-spacer adjacent motif (PAM) that is required for target recognition. After binding, a Cas protein complex serves as a DNA endonuclease to cut both strands at the target and subsequent DNA degradation occurs via exonuclease activity.

CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding and about 20 nucleotide long “spacer” or “targeting” sequence which defines the genomic target to be modified. Thus, one can change the genomic target of Cas9 by simply changing the targeting sequence present in the gRNA. Guide RNA (gRNA), as used herein refers to a synthetic fusion of the endogenous bacterial crRNA and tracrRNA, providing both targeting specificity and scaffolding/binding ability for Cas9 nuclease. Also referred to as “single guide RNA” or “sgRNA”. CRISPR was originally employed to “knock-out” target genes in various cell types and organisms, but modifications to the Cas9 enzyme have extended the application of CRISPR to “knock-in” target genes, selectively activate or repress target genes, purify specific regions of DNA, and even image DNA in live cells using fluorescence microscopy. Furthermore, the ease of generating gRNAs makes CRISPR one of the most scalable genome editing technologies and has been recently utilized for genome-wide screens.

In some embodiments, CRISPR type II system used for the method disclosed herein comprises CRISPR-associated endonuclease 9 (Cas9) and a gRNA. In some embodiments, the gRNA disclosed herein comprises a nucleotide sequence selected from: ACATGCAATACACTGAACTG (SEQ ID NO: 1), ACTGTTAACAAGGTTGGTTG (SEQ ID NO: 2), CTTGTAGGGCTTGATTACTG (SEQ ID NO: 3), TCTTGCTTTCCACTTTCCAT (SEQ ID NO: 18), AAGTGTGGAACAAACTGCTG (SEQ ID NO: 19) GATGGGCTCCACAAGGAACT (SEQ ID NO: 20), or any combination thereof. In some embodiments, the gRNA disclosed herein comprises a nucleotide sequence as set forth in SEQ ID NO: 1. In some embodiments, the gRNA comprises a nucleotide sequence as set forth in SEQ ID NO: 2. In some embodiments, the gRNA comprises a nucleotide sequence as set forth in SEQ ID NO: 3. In some embodiments, the gRNA comprises a nucleotide sequence as set forth in SEQ ID NO: 18. In some embodiments, the gRNA comprises a nucleotide sequence as set forth in SEQ ID NO: 19. In some embodiments, the gRNA comprises a nucleotide sequence as set forth in SEQ ID NO: 20.

In some embodiments, gRNA comprising a nucleotide sequence as set forth in SEQ ID NO: 1 is used for the integration of the exogenous reporter gene into site 4a. In some embodiments, gRNA comprising a nucleotide sequence as set forth in SEQ ID NO: 20 is used for the integration of the exogenous reporter gene into site 13a. In some embodiments, gRNA comprising a nucleotide sequence as set forth in SEQ ID NO: 3 is used for the integration of the exogenous reporter gene into site 14a.

In some embodiments, the recombinant Z gender chromosome comprises the nucleotide sequence:

(SEQ ID NO: 4)
GGCCTATACAGACAAATCTTTACTAAATGGAGAATCCTGACATTTTGTCCATTTCTC
TCTATACCACCAACTCTCATCTTGGTTACATTGCCTGTCAGTTGAACAGGCAAGAT
CTCCATTCCCAAAACACCACACTTATCTTGAATTGAGCCATCAAGAGGTATTTCTCT
GATACACCATTATCTAAAACCCAGTGTTATTTTTCAGTGAGGGTAGATTCCCTTTAC
TGCTTTTTCTCCTCATTTCCAGAATTCCGTTACATAACTTACGGTAAATGGCCCGCC
TGGCTGACCGCCCAACGACCCCCGCCCATTGACGTCAATAGTAACGCCAATAGGG
ACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAAT
GGCCCGCCTGGCATTGTGCCCAGTACATGACCTTATGGGACTTTCCTACTTGGCAG
TACATCTACGTATTAGTCATCGCTATTACCATGGTCGAGGTGAGCCCCACGTTCTG
CTTCACTCTCCCCATCTCCCCCCCCTCCCCACCCCCAATTTTGTATTTATTTATTTTT
TAATTATTTTGTGCAGCGATGGGGGCGGGGGGGGGGGGGGGGCGCGCGCCAGGCG
GGGCGGGGCGGGGCGAGGGGCGGGGCGGGGCGAGGCGGAGAGGTGCGGCGGCAG
CCAATCAGAGCGGCGCGCTCCGAAAGTTTCCTTTTATGGCGAGGCGGCGGCGGCG
GCGGCCCTATAAAAAGCGAAGCGCGCGGCGGGGGGAGTCGCTGCGCGCTGCCTT
CGCCCCGTGCCCCGCTCCGCCGCCGCCTCGCGCCGCCCGCCCCGGCTCTGACTGAC
CGCGTTACTCCCACAGGTGAGCGGGCGGGACGGCCCTTCTCCTCCGGGCTGTAATT
AGCTGAGCAAGAGGTAAGGGTTTAAGGGATGGTTGGTTGGTGGGGTATTAATGTTT
AATTACCTGGAGCACCTGCCTGAAATCACTTTTTTTCAGGTTGGCGCCACCATGGC
CTCCTCCGAGGACGTCATCAAGGAGTTCATGCGCTTCAAGGTGCGCATGGAGGGCT
CCGTGAACGGCCACGAGTTCGAGATCGAGGGCGAGGGCGAGGGCCGCCCCTACGA
GGGCACCCAGACCGCCAAGCTGAAGGTGACCAAGGGCGGCCCCCTGCCCTTCGCC
TGGGACATCCTGTCCCCCCAGTTCCAGTACGGCTCCAAGGTGTACGTGAAGCACCC
CGCCGACATCCCCGACTACAAGAAGCTGTCCTTCCCCGAGGGCTTCAAGTGGGAGC
GCGTGATGAACTTCGAGGACGGCGGCGTGGTGACCGTGACCCAGGACTCCTCCCT
GCAGGACGGCTCCTTCATCTACAAGGTGAAGTTCATCGGCGTGAACTTCCCCTCCG
ACGGCCCCGTAATGCAGAAGAAGACTATGGGCTGGGAGGCCTCCACCGAGCGCCT
GTACCCCCGCGACGGCGTGCTGAAGGGCGAGATCCACAAGGCCCTGAAGCTGAAG
GACGGCGGCCACTACCTGGTGGAGTTCAAGTCCATCTACATGGCCAAGAAGCCCG
TGCAGCTGCCCGGCTACTACTACGTGGACTCCAAGCTGGACATCACCTCCCACAAC
GAGGACTACACCATCGTGGAGCAGTACGAGCGCGCCGAGGGCCGCCACCACCTGT
TCCTGTAGCGGCCGCGACTCTAGATCATAATCAGCCATACCACATTTGTAGAGGTT
TTACTTGCTTTAAAAAACCTCCCACACCTCCCCCTGAACCTGAAACATAAAATGAA
TGCAATTGTTGTTGTTAACTTGTTTATTGCAGCTTATAATGGTTACAAATAAAGCAA
TAGCATCACAAATTTCACAAATAAAGCATTTTTTTCACTGCATTCTAGTTGTGGTTT
GTCCAAACTCATCAATGTATCTTAAGGCGTGGATCCATAACGGCATTCAGTACTTG
TCATCACTAATTAGTGTATTCAATCTACCCTCTATTTCTAGGACAACATGTTTTAAT
AATTCACCATAAATTAGTTCTATGAGCATTTTGATCTAGTGGTTGTTGTAGATCACA
GGATATGAATAGATTGTGTGTATGTGTAAACCAGGAGTGTCTTGACAAAAGGACA
CAGTGCTGGTAAGACCAGCAGAAGAGGGTCCCCTGTGGCAAAAATGTTTTACTGA
CCTTTG.

In some embodiments, the recombinant Z gender chromosome comprising the exogenous reporter gene in site 4a comprises the nucleotide sequence as set forth in SEQ ID NO: 4.

In some embodiments, the recombinant Z gender chromosome comprises the nucleotide sequence as set forth in SEQ ID NO: 4, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 4, comprises 50%-100%, 60%-100%, 70%-100%, 80%-100%, 90%-100%, 95%-100%, 99%-100% identity or homology to a nucleic acid sequence as set forth in SEQ ID NO: 4. Each possibility represents a separate embodiment of the invention.

In some embodiments, the recombinant Z gender chromosome comprises the nucleotide sequence:

(SEQ ID NO: 5)
AATACACAGCACTGTATCTTTTATGGCAATCTATTTTCGATTGTCTCATGTGCAGAA
CAGTTACCAGGATTGCAACAACGGATTGAATTTATCCGAAAAGAAGTTTTGTGTCC
TGCTTTGTGATAGCTGAGAAAGAAAGGCAGTGATGCTTAAAAAGCAGTCAGTGAC
CTAATCACCTACTGTCAGGTGTACTATGAATACATACAGTAGAGCCAGTAACACAG
TTTGACAGCATTTTCATTAGATGTTTGAATTCCGTTACATAACTTACGGTAAATGGC
CCGCCTGGCTGACCGCCCAACGACCCCCGCCCATTGACGTCAATAGTAACGCCAAT
AGGGACTTTCCATTGACGTCAATGGGTGGAGTATTTACGGTAAACTGCCCACTTGG
CAGTACATCAAGTGTATCATATGCCAAGTACGCCCCCTATTGACGTCAATGACGGT
AAATGGCCCGCCTGGCATTGTGCCCAGTACATGACCTTATGGGACTTTCCTACTTG
GCAGTACATCTACGTATTAGTCATCGCTATTACCATGGTCGAGGTGAGCCCCACGT
TCTGCTTCACTCTCCCCATCTCCCCCCCCTCCCCACCCCCAATTTTGTATTTATTTAT
TTTTTAATTATTTTGTGCAGCGATGGGGGCGGGGGGGGGGGGGGGGCGCGCGCCA
GGCGGGGCGGGGCGGGGCGAGGGGCGGGGCGGGGCGAGGCGGAGAGGTGCGGCG
GCAGCCAATCAGAGCGGCGCGCTCCGAAAGTTTCCTTTTATGGCGAGGCGGCGGC
GGCGGCGGCCCTATAAAAAGCGAAGCGCGCGGCGGGCGGGAGTCGCTGCGCGCTG
CCTTCGCCCCGTGCCCCGCTCCGCCGCCGCCTCGCGCCGCCCGCCCCGGCTCTGAC
TGACCGCGTTACTCCCACAGGTGAGCGGGCGGGACGGCCCTTCTCCTCCGGGCTGT
AATTAGCTGAGCAAGAGGTAAGGGTTTAAGGGATGGTTGGTTGGTGGGGTATTAA
TGTTTAATTACCTGGAGCACCTGCCTGAAATCACTTTTTTTCAGGTTGGCGCCACCA
TGGCCTCCTCCGAGGACGTCATCAAGGAGTTCATGCGCTTCAAGGTGCGCATGGAG
GGCTCCGTGAACGGCCACGAGTTCGAGATCGAGGGCGAGGGCGAGGGCCGCCCCT
ACGAGGGCACCCAGACCGCCAAGCTGAAGGTGACCAAGGGCGGCCCCCTGCCCTT
CGCCTGGGACATCCTGTCCCCCCAGTTCCAGTACGGCTCCAAGGTGTACGTGAAGC
ACCCCGCCGACATCCCCGACTACAAGAAGCTGTCCTTCCCCGAGGGCTTCAAGTGG
GAGCGCGTGATGAACTTCGAGGACGGCGGCGTGGTGACCGTGACCCAGGACTCCT
CCCTGCAGGACGGCTCCTTCATCTACAAGGTGAAGTTCATCGGCGTGAACTTCCCC
TCCGACGGCCCCGTAATGCAGAAGAAGACTATGGGCTGGGAGGCCTCCACCGAGC
GCCTGTACCCCCGCGACGGCGTGCTGAAGGGCGAGATCCACAAGGCCCTGAAGCT
GAAGGACGGCGGCCACTACCTGGTGGAGTTCAAGTCCATCTACATGGCCAAGAAG
CCCGTGCAGCTGCCCGGCTACTACTACGTGGACTCCAAGCTGGACATCACCTCCCA
CAACGAGGACTACACCATCGTGGAGCAGTACGAGCGCGCCGAGGGCCGCCACCAC
CTGTTCCTGTAGCGGCCGCGACTCTAGATCATAATCAGCCATACCACATTTGTAGA
GGTTTTACTTGCTTTAAAAAACCTCCCACACCTCCCCCTGAACCTGAAACATAAAA
TGAATGCAATTGTTGTTGTTAACTTGTTTATTGCAGCTTATAATGGTTACAAATAAA
GCAATAGCATCACAAATTTCACAAATAAAGCATTTTTTTCACTGCATTCTAGTTGTG
GTTTGTCCAAACTCATCAATGTATCTTAAGGCGTGGATCCCAGAACAAAATAGTAT
TTTTGTTCACAACTGGGAGTGAAATCTGATTTCAAACCACTAAAAAGAATAGTGGA
GACATGAAGAAAAAACGTTTTGTCTGAATGCTTTCTTGGGTAGTCAGAAATAAAAG
CTGTTGTACGGAAGATCATATGAGGCTGCTATGGGTAGCAGCATCAAGTGTGGCAG
TGGAGCAGAGAGAGATTGCATGCCACGGGGAGAGGAAATGTGGAAAATTACACAT
ATCACCGTGAG.

In some embodiments, the recombinant Z gender chromosome comprising the exogenous reporter gene in site 5a comprises the nucleotide sequence as set forth in SEQ ID NO: 5.

In some embodiments, the recombinant Z gender chromosome comprises the nucleotide sequence as set forth in SEQ ID NO: 5, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 5, comprises 50%-100%, 60%-100%, 70%-100%, 80%-100%, 90%-100%, 95%-100%, 99%-100% identity or homology to a nucleic acid sequence as set forth in SEQ ID NO: 5. Each possibility represents a separate embodiment of the invention.

In some embodiments, the recombinant Z gender chromosome comprises the nucleotide sequence:

(SEQ ID NO: 6)
GGTCTGTGCAAACAGTGTTTCTCATGCAACTTGGCTGCCTTTAAATGGCTTACCAAC
TCTTTTCTGAAAAACCTAAAAAATGTCTGTGTGCCAAGTAAGATACTTCAATTCAA
AAGGAGGTTTTTCAATTTTTTCTCTCAGTTTATCATTTCTTCTACTTGAAAAATATAT
TTTAATTTTAATGTTTTTTGTTTTATACAAAAATATATGAACTATGTATTACTATTGT
CACCTGTCATTACCAAGAAGTGAATTCCGTTACATAACTTACGGTAAATGGCCCGC
CTGGCTGACCGCCCAACGACCCCCGCCCATTGACGTCAATAGTAACGCCAATAGG
GACTTTCCATTGACGTCAATGGGTGGAGTATTTACGGTAAACTGCCCACTTGGCAG
TACATCAAGTGTATCATATGCCAAGTACGCCCCCTATTGACGTCAATGACGGTAAA
TGGCCCGCCTGGCATTGTGCCCAGTACATGACCTTATGGGACTTTCCTACTTGGCA
GTACATCTACGTATTAGTCATCGCTATTACCATGGTCGAGGTGAGCCCCACGTTCT
GCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGC
GGGGCGGGGCGGGGCGAGGGGCGGGGCGGGGCGAGGCGGAGAGGTGCGGCGGCA
GCCAATCAGAGCGGCGCGCTCCGAAAGTTTCCTTTTATGGCGAGGCGGCGGCGGC
GGCGGCCCTATAAAAAGCGAAGCGCGCGGCGGGCGGGAGTCGCTGCGCGCTGCCT
TCGCCCCGTGCCCCGCTCCGCCGCCGCCTCGCGCCGCCCGCCCCGGCTCTGACTGA
CCGCGTTACTCCCACAGGTGAGCGGGGGGACGGCCCTTCTCCTCCGGGCTGTAAT
TAGCTGAGCAAGAGGTAAGGGTTTAAGGGATGGTTGGTTGGTGGGGTATTAATGTT
TAATTACCTGGAGCACCTGCCTGAAATCACTTTTTTTCAGGTTGGCGCCACCATGG
CCTCCTCCGAGGACGTCATCAAGGAGTTCATGCGCTTCAAGGTGCGCATGGAGGGC
TCCGTGAACGGCCACGAGTTCGAGATCGAGGGCGAGGGCGAGGGCCGCCCCTACG
AGGGCACCCAGACCGCCAAGCTGAAGGTGACCAAGGGCGGCCCCCTGCCCTTCGC
CTGGGACATCCTGTCCCCCCAGTTCCAGTACGGCTCCAAGGTGTACGTGAAGCACC
CCGCCGACATCCCCGACTACAAGAAGCTGTCCTTCCCCGAGGGCTTCAAGTGGGAG
CGCGTGATGAACTTCGAGGACGGCGGCGTGGTGACCGTGACCCAGGACTCCTCCCT
GCAGGACGGCTCCTTCATCTACAAGGTGAAGTTCATCGGCGTGAACTTCCCCTCCG
ACGGCCCCGTAATGCAGAAGAAGACTATGGGCTGGGAGGCCTCCACCGAGCGCCT
GTACCCCCGCGACGGCGTGCTGAAGGGCGAGATCCACAAGGCCCTGAAGCTGAAG
GACGGCGGCCACTACCTGGTGGAGTTCAAGTCCATCTACATGGCCAAGAAGCCCG
TGCAGCTGCCCGGCTACTACTACGTGGACTCCAAGCTGGACATCACCTCCCACAAC
GAGGACTACACCATCGTGGAGCAGTACGAGCGCGCCGAGGGCCGCCACCACCTGT
TCCTGTAGCGGCCGCGACTCTAGATCATAATCAGCCATACCACATTTGTAGAGGTT
TTACTTGCTTTAAAAAACCTCCCACACCTCCCCCTGAACCTGAAACATAAAATGAA
TGCAATTGTTGTTGTTAACTTGTTTATTGCAGCTTATAATGGTTACAAATAAAGCAA
TAGCATCACAAATTTCACAAATAAAGCATTTTTTTCACTGCATTCTAGTTGTGGTTT
GTCCAAACTCATCAATGTATCTTAAGGCGTGGATCCACATCACCAGGGCTGGAGGT
TACATCCTGCTGGCCAAAACTACATTATGGTGGGTTAGCTAATTTTACAGGAGGGA
TAAATTGTGAGAATCTGGAAACCATAATCTTGACAAGAAAAAATTAACACCCAATT
CCTTGGTGAGACTGGGCATTATATGGACATGGGAAATCTCACAGTCATGATATATG
TTAGGAAGGAACTCTCCTTCAAGGGTCCAGGACTGTAAGTATTGGCCTGCCCCAAC
ACTTGG

In some embodiments, the recombinant Z gender chromosome comprising the exogenous reporter gene in site 14a comprises the nucleotide sequence as set forth in SEQ ID NO: 6.

In some embodiments, the recombinant Z gender chromosome comprises the nucleotide sequence as set forth in SEQ ID NO: 6, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 6, comprises 50%-100%, 60%-100%, 70%-100%, 80%-100%, 90%-100%, 95%-100%, 99%-100% identity or homology to a nucleic acid sequence as set forth in SEQ ID NO: 6. Each possibility represents a separate embodiment of the invention.

In some embodiments, the recombinant Z gender chromosome comprises the nucleotide sequence:

(SEQ ID NO: 39)
TGAATGTGGAAAGTGAAGATAACTACTAGTGCAGCAAGCAGACATAGAGATTTTA
TAAAAGTGGATGGTGAGCATTGTTTGTTAGATACAAACCATTAAGCAGAGATGCCT
AGTGACTCAGAGTACAGAGCTTCCTGACTGTCAACACTGACCATGACACTGACCAC
GTTATGATCCAGGAATAATAGAACCTGTATTTACTCTTAGATATTCTTAAATTGTAA
TCACAGAAGAGGACAAACAGTGATAGGAATTCCGTTACATAACTTACGGTAAATG
GCCCGCCTGGCTGACCGCCCAACGACCCCCGCCCATTGACGTCAATAGTAACGCCA
ATAGGGACTTTCCATTGACGTCAATGGGTGGAGTATTTACGGTAAACTGCCCACTT
GGCAGTACATCAAGTGTATCATATGCCAAGTACGCCCCCTATTGACGTCAATGACG
GTAAATGGCCCGCCTGGCATTGTGCCCAGTACATGACCTTATGGGACTTTCCTACT
TGGCAGTACATCTACGTATTAGTCATCGCTATTACCATGGTCGAGGTGAGCCCCAC
GTTCTGCTTCACTCTCCCCATCTCCCCCCCCTCCCCACCCCCAATTTTGTATTTATTT
ATTTTTTAATTATTTTGTGCAGCGATGGGGGCGGGGGGGGGGGGGGGGCGCGCGC
CAGGCGGGGCGGGGCGGGGCGAGGGGCGGGGCGGGGCGAGGCGGAGAGGTGCGG
CGGCAGCCAATCAGAGCGGCGCGCTCCGAAAGTTTCCTTTTATGGCGAGGCGGCG
GCGGCGGCGGCCCTATAAAAAGCGAAGCGCGCGGCGGGCGGGAGTCGCTGCGCGC
TGCCTTCGCCCCGTGCCCCGCTCCGCCGCCGCCTCGCGCCGCCCGCCCCGGCTCTG
ACTGACCGCGTTACTCCCACAGGTGAGCGGGCGGGACGGCCCTTCTCCTCCGGGCT
GTAATTAGCTGAGCAAGAGGTAAGGGTTTAAGGGATGGTTGGTTGGTGGGGTATT
AATGTTTAATTACCTGGAGCACCTGCCTGAAATCACTTTTTTTCAGGTTGGCGCCAC
CATGGCCTCCTCCGAGGACGTCATCAAGGAGTTCATGCGCTTCAAGGTGCGCATGG
AGGGCTCCGTGAACGGCCACGAGTTCGAGATCGAGGGCGAGGGCGAGGGCCGCCC
CTACGAGGGCACCCAGACCGCCAAGCTGAAGGTGACCAAGGGCGGCCCCCTGCCC
TTCGCCTGGGACATCCTGTCCCCCCAGTTCCAGTACGGCTCCAAGGTGTACGTGAA
GCACCCCGCCGACATCCCCGACTACAAGAAGCTGTCCTTCCCCGAGGGCTTCAAGT
GGGAGCGCGTGATGAACTTCGAGGACGGCGGCGTGGTGACCGTGACCCAGGACTC
CTCCCTGCAGGACGGCTCCTTCATCTACAAGGTGAAGTTCATCGGCGTGAACTTCC
CCTCCGACGGCCCCGTAATGCAGAAGAAGACTATGGGCTGGGAGGCCTCCACCGA
GCGCCTGTACCCCCGCGACGGCGTGCTGAAGGGCGAGATCCACAAGGCCCTGAAG
CTGAAGGACGGCGGCCACTACCTGGTGGAGTTCAAGTCCATCTACATGGCCAAGA
AGCCCGTGCAGCTGCCCGGCTACTACTACGTGGACTCCAAGCTGGACATCACCTCC
CACAACGAGGACTACACCATCGTGGAGCAGTACGAGCGCGCCGAGGGCCGCCACC
ACCTGTTCCTGTAGCGGCCGCGACTCTAGATCATAATCAGCCATACCACATTTGTA
GAGGTTTTACTTGCTTTAAAAAACCTCCCACACCTCCCCCTGAACCTGAAACATAA
AATGAATGCAATTGTTGTTGTTAACTTGTTTATTGCAGCTTATAATGGTTACAAATA
AAGCAATAGCATCACAAATTTCACAAATAAAGCATTTTTTTCACTGCATTCTAGTT
GTGGTTTGTCCAAACTCATCAATGTATCTTAAGGCGTGGATCCTCTATTGTCTCATA
AGTCCTACCACTCAGGAATAAGGCTTATAAGAAACATGAGAAATGTGTAACTGTA
GAGTGAGTTAGACATAGCCAGCAGCTATATACGTACCCATATGCTCACAGTCTCTC
TCCTTCGCAGGATTAGGAAATAAAATTGGGTGAAAAAGGTTATAGATAAGACAGA
GATAGAAAGATTGCTAATCATTTAACATATTTGATTTGGGCAAAGTAATTGAATAC
ACTGTCAAGTAAAG.

In some embodiments, the recombinant Z gender chromosome comprising the exogenous reporter gene in site 7b comprises the nucleotide sequence as set forth in SEQ ID NO: 39.

In some embodiments, the recombinant Z gender chromosome comprises the nucleotide sequence as set forth in SEQ ID NO: 39, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 39, comprises 50%-100%, 60%-100%, 70%-100%, 80%-100%, 90%-100%, 95%-100%, 99%-100% identity or homology to a nucleic acid sequence as set forth in SEQ ID NO: 39. Each possibility represents a separate embodiment of the invention.

In some embodiments, the recombinant Z gender chromosome comprises the nucleotide sequence:

(SEQ ID NO: 40)
CAAGAACATTAAAAAGAAACAGGATCTCTCTTGCTTTTGTTAAGAAAAAAAAACA
GCAGCAGTACACCAAGAGCAGTTAAATTACTCTATTTAGAGATGTCTGAAACACTT
CTAAAGCAAAGCAGGTAATACTTCAGGGACCATCTACAAGCTTGCAGTTTTAGCTC
CCTTCTATAACTTTTCCAATTTGATCTTTCTGTGGAAAAATACAAAATTGTGTCTGT
TTCTTGAAGACCGGTAACCAGAAAGTGAATTCCGTTACATAACTTACGGTAAATGG
CCCGCCTGGCTGACCGCCCAACGACCCCCGCCCATTGACGTCAATAGTAACGCCAA
TAGGGACTTTCCATTGACGTCAATGGGTGGAGTATTTACGGTAAACTGCCCACTTG
GCAGTACATCAAGTGTATCATATGCCAAGTACGCCCCCTATTGACGTCAATGACGG
TAAATGGCCCGCCTGGCATTGTGCCCAGTACATGACCTTATGGGACTTTCCTACTT
GGCAGTACATCTACGTATTAGTCATCGCTATTACCATGGTCGAGGTGAGCCCCACG
TTCTGCTTCACTCTCCCCATCTCCCCCCCCTCCCCACCCCCAATTTTGTATTTATTTA
TTTTTTAATTATTTTGTGCAGCGATGGGGGCGGGGGGGGGGGGGGGGCGCGCGCC
AGGCGGGGCGGGGCGGGGCGAGGGGCGGGGCGGGGCGAGGCGGAGAGGTGCGGC
GGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGCGCT
GCCTTCGCCCCGTGCCCCGCTCCGCCGCCGCCTCGCGCCGCCCGCCCCGGCTCTGA
CTGACCGCGTTACTCCCACAGGTGAGCGGGCGGGACGGCCCTTCTCCTCCGGGCTG
TAATTAGCTGAGCAAGAGGTAAGGGTTTAAGGGATGGTTGGTTGGTGGGGTATTA
ATGTTTAATTACCTGGAGCACCTGCCTGAAATCACTTTTTTTCAGGTTGGCGCCACC
ATGGCCTCCTCCGAGGACGTCATCAAGGAGTTCATGCGCTTCAAGGTGCGCATGGA
GGGCTCCGTGAACGGCCACGAGTTCGAGATCGAGGGCGAGGGCGAGGGCCGCCCC
TACGAGGGCACCCAGACCGCCAAGCTGAAGGTGACCAAGGGCGGCCCCCTGCCCT
TCGCCTGGGACATCCTGTCCCCCCAGTTCCAGTACGGCTCCAAGGTGTACGTGAAG
CACCCCGCCGACATCCCCGACTACAAGAAGCTGTCCTTCCCCGAGGGCTTCAAGTG
GGAGCGCGTGATGAACTTCGAGGACGGCGGCGTGGTGACCGTGACCCAGGACTCC
TCCCTGCAGGACGGCTCCTTCATCTACAAGGTGAAGTTCATCGGCGTGAACTTCCC
CTCCGACGGCCCCGTAATGCAGAAGAAGACTATGGGCTGGGAGGCCTCCACCGAG
CGCCTGTACCCCCGCGACGGCGTGCTGAAGGGCGAGATCCACAAGGCCCTGAAGC
TGAAGGACGGCGGCCACTACCTGGTGGAGTTCAAGTCCATCTACATGGCCAAGAA
GCCCGTGCAGCTGCCCGGCTACTACTACGTGGACTCCAAGCTGGACATCACCTCCC
ACAACGAGGACTACACCATCGTGGAGCAGTACGAGCGCGCCGAGGGCCGCCACCA
CCTGTTCCTGTAGCGGCCGCGACTCTAGATCATAATCAGCCATACCACATTTGTAG
AGGTTTTACTTGCTTTAAAAAACCTCCCACACCTCCCCCTGAACCTGAAACATAAA
ATGAATGCAATTGTTGTTGTTAACTTGTTTATTGCAGCTTATAATGGTTACAAATAA
AGCAATAGCATCACAAATTTCACAAATAAAGCATTTTTTTCACTGCATTCTAGTTGT
GGTTTGTCCAAACTCATCAATGTATCTTAAGGCGTGGATCCTATTTGTCAGAAAAA
TCTATCTGCATACTCATTTCTTTGAATGAGATTACAATCATGAGATGTCCACTATCT
GCATTTTTGATCACTAGTGAAAACTCCTTCTTTCCAAAGCCACTGGTGATTATTGTT
TATTTAGGAGAGCAGGCCTTTAAAAGAAATACTGTGGTCACCTGTGTGACTGAAAA
AAGCACATATTCAATTCATGTGAATAACATTAGAAAGTTTCCAGGAACTGCTGATG
ATTCAAGCA.

In some embodiments, the recombinant Z gender chromosome comprising the exogenous reporter gene in site 8a comprises the nucleotide sequence as set forth in SEQ ID NO: 40.

In some embodiments, the recombinant Z gender chromosome comprises the nucleotide sequence as set forth in SEQ ID NO: 40, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 40, comprises 50%-100%, 60%-100%, 70%-100%, 80%-100%, 90%-100%, 95%-100%, 99%-100% identity or homology to a nucleic acid sequence as set forth in SEQ ID NO: 40. Each possibility represents a separate embodiment of the invention.

In some embodiments, the recombinant Z gender chromosome comprises the nucleotide sequence:

(SEQ ID NO: 41)
GAAACATCATTTATAGACTAAGTGTGTATATTATAAATCTGCTGATTGCATAGGTA
AATTTTTTTCAGTATCATCTGCTGTCAAAATTTTGGCTGTGATGAAATCAACTTTCT
TCATAGAAACTCATATAATGCTGTGCTTCGGATTTTTGATGAAAATAGTGATGATA
ATACACCAGTGTTCCAGTTGTGACAGAGCAGTGCTTACAGAGTCAAAAACTTTTTA
TTTTATTGTGCTATCCTGCCAATGAGAATTCCGTTACATAACTTACGGTAAATGGCC
CGCCTGGCTGACCGCCCAACGACCCCCGCCCATTGACGTCAATAGTAACGCCAATA
GGGACTTTCCATTGACGTCAATGGGTGGAGTATTTACGGTAAACTGCCCACTTGGC
AGTACATCAAGTGTATCATATGCCAAGTACGCCCCCTATTGACGTCAATGACGGTA
AATGGCCCGCCTGGCATTGTGCCCAGTACATGACCTTATGGGACTTTCCTACTTGG
CAGTACATCTACGTATTAGTCATCGCTATTACCATGGTCGAGGTGAGCCCCACGTT
CTGCTTCACTCTCCCCATCTCCCCCCCCTCCCCACCCCCAATTTTGTATTTATTTATT
TTTTAATTATTTTGTGCAGCGATGGGGGCGGGGGGGGGGGGGGGGCGCGCGCCAG
GCGGGGCGGGGCGGGGCGAGGGGCGGGGCGGGGCGAGGCGGAGAGGTGCGGCGG
CAGCCAATCAGAGCGGCGCGCTCCGAAAGTTTCCTTTTATGGCGAGGCGGCGGCG
GCGGCGGCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGCTCTGACT
GACCGCGTTACTCCCACAGGTGAGCGGGCGGGACGGCCCTTCTCCTCCGGGCTGTA
ATTAGCTGAGCAAGAGGTAAGGGTTTAAGGGATGGTTGGTTGGTGGGGTATTAAT
GTTTAATTACCTGGAGCACCTGCCTGAAATCACTTTTTTTCAGGTTGGCGCCACCAT
GGCCTCCTCCGAGGACGTCATCAAGGAGTTCATGCGCTTCAAGGTGCGCATGGAG
GGCTCCGTGAACGGCCACGAGTTCGAGATCGAGGGCGAGGGCGAGGGCCGCCCCT
ACGAGGGCACCCAGACCGCCAAGCTGAAGGTGACCAAGGGCGGCCCCCTGCCCTT
CGCCTGGGACATCCTGTCCCCCCAGTTCCAGTACGGCTCCAAGGTGTACGTGAAGC
ACCCCGCCGACATCCCCGACTACAAGAAGCTGTCCTTCCCCGAGGGCTTCAAGTGG
GAGCGCGTGATGAACTTCGAGGACGGCGGCGTGGTGACCGTGACCCAGGACTCCT
CCCTGCAGGACGGCTCCTTCATCTACAAGGTGAAGTTCATCGGCGTGAACTTCCCC
TCCGACGGCCCCGTAATGCAGAAGAAGACTATGGGCTGGGAGGCCTCCACCGAGC
GCCTGTACCCCCGCGACGGCGTGCTGAAGGGCGAGATCCACAAGGCCCTGAAGCT
GAAGGACGGCGGCCACTACCTGGTGGAGTTCAAGTCCATCTACATGGCCAAGAAG
CCCGTGCAGCTGCCCGGCTACTACTACGTGGACTCCAAGCTGGACATCACCTCCCA
CAACGAGGACTACACCATCGTGGAGCAGTACGAGCGCGCCGAGGGCCGCCACCAC
CTGTTCCTGTAGCGGCCGCGACTCTAGATCATAATCAGCCATACCACATTTGTAGA
GGTTTTACTTGCTTTAAAAAACCTCCCACACCTCCCCCTGAACCTGAAACATAAAA
TGAATGCAATTGTTGTTGTTAACTTGTTTATTGCAGCTTATAATGGTTACAAATAAA
GCAATAGCATCACAAATTTCACAAATAAAGCATTTTTTTCACTGCATTCTAGTTGTG
GTTTGTCCAAACTCATCAATGTATCTTAAGGCGTGGATCCTGCTTTGCTTGCACATG
CAGTAAACTTTTTTAATCTTAATTCATAAACTCTCACACTTTTACATTTCCAATTTTC
ACATCTTTAATCATCTGGGAGATGATTTCACATCCTCATCAAGTTTGTGGATGACCC
AGAACTGAGGGCAGTGGCTAACTCACCAGAGTGCTGTGCTGCCATCTGGAGGAAC
TTGCACAGTTGGAGAAGGGCTGACAGGAACTTGATGGAGTTCTACATGGAGAAGG
GAAAGTTC.

In some embodiments, the recombinant Z gender chromosome comprising the exogenous reporter gene in site 13a comprises the nucleotide sequence as set forth in SEQ ID NO: 41.

In some embodiments, the recombinant Z gender chromosome comprises the nucleotide sequence as set forth in SEQ ID NO: 41, or an analog thereof, having at least 50%, 60%, 70%, 80%, 90%, 95%, 99% identity or homology thereto, or any value and range therebetween. Each possibility represents a separate embodiment of the invention. In some embodiments, an analog of the nucleotide sequence as set forth in SEQ ID NO: 41, comprises 50%-100%, 60%-100%, 70%-100%, 80%-100%, 90%-100%, 95%-100%, 99%-100% identity or homology to a nucleic acid sequence as set forth in SEQ ID NO: 41. Each possibility represents a separate embodiment of the invention.

According to another aspect, there is provided a cell obtained or derived from the transgenic female chicken disclosed herein. In some embodiments, the cell comprises a recombinant Z chromosome, as disclosed herein. In some embodiments, the exogenous reporter gene, encoding RFP, is integrated into the Z chromosome of the cell. In some embodiments, the cell comprises a primordial germ cell (PGC). In some embodiments, the cell is PGC. In some embodiments, there is provided a PGC comprising a nucleic acid sequence of an exogenous reporter gene encoding RFP, integrated into the Z gender chromosome thereof.

The term “germ cell” refers to an embryonic cell with a potential of developing into a gamete. The term “Primordial germ cell (PGC)”, as used herein, relates to a germline stem cell that serves as a progenitor of a gamete and gives rise to a pluripotent embryonic stem cell.

The use of PGCs to produce transgenic chickens is well-known in the art. PGCs at an early stage are readily accessible and can be manipulated in vitro for practical applications, including the restoration of genetic material and genome editing. The capability of chicken PGCs to be maintained undifferentiated in vitro without losing their properties, as well as their highly migratory capacity via the bloodstream of the chicken embryo, highlight PGCs as a leading source for transgenesis. In some embodiments, PGCs are used to produce a chimeric and/or a transgenic chicken via the injection of genetically manipulated PGCs into the blood vessels of a recipient egg. However, it is noted that other cells are known in the art for generation of a transgenic chicken. Non-limiting examples for such cells include embryonic stem cells (ESC) and spermatogonial stem cells (SSC).

According to another aspect there is provided a method for producing a chicken comprising a recombinant Z gender chromosome, the method comprising: (a) obtaining at least one transgenic PGC comprising a recombinant Z chromosome comprising a nucleic acid sequence of an exogenous reporter gene encoding an RFP in at least one location as listed under Table 1 in Z gender chromosome of a chicken; and, (b) transplanting the transgenic PGC into a receptive chicken embryo, thereby, producing a chicken comprising the recombinant Z gender chromosome.

In some embodiments, transplanting comprises injecting the transgenic PGCs into at least one blood vessel of a recipient egg.

In some embodiments, the recombinant Z chromosome comprises a nucleic acid sequence of an exogenous reporter gene encoding an RFP in at least one location as listed under Table 3. In some embodiments, the at least one location is selected from: site 4a, site 5a, site 13a, site 14a, or any combination thereof.

In some embodiments, the method disclosed herein is for producing a chimeric or a transgenic chicken. It is noted that steps (a) and (b) as disclosed herein merely refer to the necessary steps to obtain a chicken comprising recombinant Z gender chromosome, such as, but not limited to a chimeric chicken. Other steps applicable for the generation of a transgenic chicken, and protocols thereof (e.g., protocols for germline chimera generation and screening techniques for the presence of the recombinant Z chromosome) are well known in the art. Some of these protocols are disclosed in WO2017094015A1 which is incorporated herein by reference in its entirety.

In some embodiments, the method further comprises a step preceding step (a), comprising isolating at least one PGC from the blood of a chick embryo. In one embodiment, a chick embryo is a two days-old embryo. In some embodiments, a chick embryo is a naïve chick embryo. In some embodiments, a chick embryo is a naïve two days-old chick embryo.

As used herein, the term “naïve” refers to an embryo having a genome being devoid of a recombinant Z gender chromosome as disclosed herein. In some embodiments, naïve refers to an embryo having a genome being devoid of a nucleic acid sequence of an exogenous reporter gene encoding RFP as disclosed herein.

In some embodiments, the method further comprises a step preceding step (a), comprising integrating the nucleic acid sequence of an exogenous reporter gene encoding an RFP into at least one location as listed under Table 1, in Z gender chromosome of the PGC, thereby obtaining a transgenic PGC. In some embodiments, integrating the nucleic acid sequence of an exogenous reporter gene encoding RFP as disclosed herein is performed by using a CRISPR/Cas9 system. In some embodiments, integrating the nucleic acid sequence of an exogenous reporter gene encoding RFP is performed by using CRISPR/Cas9 system comprising gRNA, comprising a nucleic acid sequence as set forth in any one of SEQ ID Nos: 1-3, and 18-20.

In some embodiments, transplanting the transgenic PGC into a receptive chicken embryo of step (b), comprises injecting the PGC into the circulatory system of the receptive chicken embryo.

In some embodiments, the method for producing a chicken comprising a recombinant Z gender chromosome further comprises one or more steps selected from: (c) incubating the embryo transplanted with the PGC at 36-38° C. until hatching; (d) rearing a chick developing from the transplanted embryo of step (c) to sexual maturity; (e) crossing the sexually mature chicken developing from the chick of step (d) with a corresponding counterpart; (e) isolating at least one offspring from a progeny obtained by the crossing of step (e) which comprises the recombinant Z gender chromosome, or any combination thereof. In some embodiments, crossing comprises at least one crossing. In some embodiments, crossing comprises plurality of crossing steps to generate a transgenic chicken disclosed herein.

In some embodiments, the transgenic PGC comprising a recombinant Z chromosome is a female PGC e.g., obtained from a female chick embryo. In some embodiments, the transgenic female PGC is transplanted into a receptive female chicken embryo, thereby producing a female chicken comprising the recombinant Z gender chromosome. In some embodiments, a chick developing from the transplanted embryo is a chimeric female chick, comprising the recombinant Z gender chromosome. In some embodiments, the chimeric female chicken is further crossed with a corresponding male counterpart. In some embodiments, the corresponding male counterpart is a wild-type male.

In some embodiments, the transgenic PGC comprising a recombinant Z chromosome is a male PGC, e.g., obtained from a male chick embryo. In some embodiments, the transgenic male PGC is transplanted into a receptive male chicken embryo, thereby, producing a male chicken comprising the recombinant Z gender chromosome. In some embodiments, a chick developing from the transplanted embryo is a chimeric male chick, comprising the recombinant Z gender chromosome. In some embodiments, the chimeric male chicken is further crossed with a corresponding female counterpart. In some embodiments, the corresponding female counterpart is a wild-type female.

In some embodiments, there is provided a method for producing a male chicken comprising at least one recombinant Z gender chromosome as disclosed herein, the method comprising obtaining at least one male progeny from the chimeric male or female chicken disclosed herein. In some embodiments, the male chick comprises two recombinant Z gender chromosomes. In some embodiments, the method further comprises a step comprising isolating or selecting a male chick comprising two recombinant Z gender chromosomes, as disclosed herein. In some embodiments, the method further comprises obtaining at least one female offspring from a male chicken comprising two recombinant Z gender chromosomes, as disclosed herein, thereby obtaining a transgenic female chicken comprising a recombinant Z gender chromosome, as disclosed herein.

### Method of Gender Determination

According to another aspect there is provided a method of gender determination of a chicken embryo in an unhatched egg comprising the embryo within a structurally integral shell, the method comprising: (a) obtaining at least one unhatched egg comprising an embryo within a structurally integral shell, from a transgenic female chicken as disclosed herein; and (b) determining whether a red fluorescent signal is detected in the embryo residing in the unhatched egg.

In some embodiments, detection of the red fluorescent signal indicates the expression of RFP in the embryo within the structurally integral shell of the unhatched egg, and thus indicates the presence of the recombinant Z chromosome in the embryo, thereby determining that the chicken embryo in the unhatched egg is a male embryo.

In some embodiments, no detection of red fluorescent signal in the embryo residing in the unhatched egg indicates that RFP is not expressed in the embryo, and thus indicates the absence of the recombinant Z chromosome in the embryo, thereby determining that the chicken embryo in the unhatched egg is a female embryo.

In some embodiments, a transgenic female chicken comprises a recombinant Z gender chromosome comprising the nucleotide sequence set forth in any one of SEQ ID Nos: 4-6, and 39-41, or an analog thereof, as disclosed herein.

In some embodiments, the method further comprises a step proceeding step (b), comprising isolating at least one unhatched egg determined to be comprising a female embryo, e.g., a red fluorescent signal is not detected, thus isolating at least one female embryo.

As used herein, the term “structurally integral shell” refers to a shell of an egg, not being: structurally damaged, cracked, broken, hatched, punched, pierced, thinned, or any combination thereof. It should be appreciated that the method of gender determination disclosed herein may be applicable for unhatched eggs of any embryonic development stage of a chicken embryo.

The term “embryonic development stage of a chicken embryo”, as used herein, refers to the stage of day 1 wherein the germinal disc is at the blastodermal stage and the segmentation cavity takes on the shape of a dark ring; the stage of day 2 wherein the first groove appears at the center of the blastoderm and the vitelline membrane appears; the stage of day 3 wherein blood circulation starts, the head and trunk can be discerned, as well as the brain and the cardiac structures which begins to beat; the stage of day 4 wherein the amniotic cavity is developing to surround the embryo and the allantoic vesicle appears; the stage of day 5 wherein the embryo takes a C shape and limbs are extending; the stage of day 6 wherein fingers of the upper and lower limbs becomes distinct; the stage of day 7 wherein the neck clearly separates the head from the body, the beak is formed and the brain progressively enters the cephalic region; the stage of day 8 wherein eye pigmentation is readily visible, the wings and legs are differentiated and the external auditory canal is opening; the stage of day 9 wherein claws appears and the first feather follicles are budding; the stage of day 10 wherein the nostrils are present, eyelids grow and the egg-tooth appears; the stage of day 11 wherein the palpebral aperture has an elliptic shape and the embryo has the aspect of a chick; the stage of day 12 wherein feather follicles surround the external auditory meatus and cover the upper eyelid whereas the lower eyelid covers major part of the cornea; the stage of day 13 wherein the allantois becomes the chorioallantoic membrane while claws and leg scales becomes apparent; the stage of days 14 to 16 wherein the whole body grows rapidly, vitellus shrinking accelerates and the egg white progressively disappears; the stage of day 17 wherein the renal system produces urates, the beak points to the air cell and the egg white is fully resorbed; the stage of day 18 wherein the vitellus internalized and the amount of amniotic fluid is reduced; the stage of day 19 wherein vitellus resorption accelerates and the beak is ready to pierce the inner shell membrane; the stage of day 20 wherein the vitellus is fully resorbed, the umbilicus is closed, the chick pierces the inner shell membrane, breathes in the air cell and is ready to hatch; the stage of day 21 wherein the chick pierces the shell in a circular way by means of its egg-tooth, extricates itself from the shell in 12 to 18 hours and lets its down dry off.

In some embodiments, the method comprises determining the gender of a chicken embryo in-ovo, inside an intact egg, while residing in an egg having an integral or intact shell, or any combination thereof, at every stage of the embryonic developmental process. In some embodiments, the method comprises determining the gender of a chicken embryo from day 1 to day 21, from day 1 to day 20, from day 1 to day 19, from day 1 to day 18, from day 1 to day 17, from day 1 to day 16, from day 1 to day 15, from day 1 to day 14, from day 1 to day 13, from day 1 to day 12, from day 1 to day 10, from day 1, to day 9, from day 1 to day 8, from day 1 to day 7, from day 1 to day 6, and from day 1 to day 5. Each possibility represents a separate embodiment of the present invention.

In some embodiments, the method further comprises a step comprising subjecting the unhatched egg comprising the embryo within a structurally integral shell to a light source.

In some embodiments, the light source is applicable or configured to detect RFP. In some embodiments, the light source comprises a wavelength of between about 400 to about 650. In some embodiments, the light source comprises a wavelength of between 500 nm and about 650 nm. In some embodiments, the light source comprises a wavelength ranging between about 515 to about 555. In some embodiments, the light source comprises a wavelength range of 500-600, 510-600, 520-600, 520-600, 530-600, 500-590, 510-590, 520-590, 530-590, 500-580, 510-580, 520-580, 530-580, 500-570, 510-570, 520-570, 530-570, 500-560, 510-560, 520-560, 530-560, 500-550, 510-550, 520-550, 530-550, 500-540, 510-540, 510-540, 520-540, or 530-540 nm. Each possibility represents a separate embodiment of the invention. In yet some further embodiments, the wavelength may be about 532 nm.

In some specific and non-limiting embodiment, the light source may be provided by a laser.

As used herein, the term “laser” refers to an electromagnetic radiation of any frequency that is amplified by stimulated emission of radiation. A laser also refers to a device that stimulates atoms or molecules to emit light at particular wavelengths and amplifies that light, typically producing a very narrow beam of radiation. In some embodiments, the light source is a green laser.

In some embodiments, the unhatched egg is exposed to the light source. In some embodiments, the egg is placed in a position enabling the exposure of the embryo at any stage to the light source. In some embodiments, a region containing the upper face of the egg yolk at stage X of the egg is excited with the light source.

In some further embodiments, the step of subjecting the unhatched egg to a light source is provided by a system, apparatus or a device that may comprise a laser source, a stand for the egg, a lens, a filter, a stand for a detector and a detector.

As used herein, the term “detector” refers to any type of device that detects and/or measures light. In some embodiments, it should be noted that the detectable signal, specifically, the fluorescent signal may be detected using suitable fluorescent means. In some embodiments, the detectable signal formed by the exogenous RFP reporter gene may be detected by light sensitive apparatus such as modified optical microscopes or Charge Coupled Device (CCD), a highly sensitive photon detector.

It is noted that, in some embodiments, the method further comprises a step, a system or device for performing same, required for RFP detection in the examined unhatched egg. Examples for such steps, systems, and/or devices are further described in WO2017094015A1, which is incorporated herein by reference in its entirety.

### Kits

According to another aspect, there is provided a kit comprising at least one first nucleic acid molecule encoding a guide RNA (gRNA), comprising the nucleotide sequence as set forth in any one of SEQ ID Nos: 1-3, and 18-20.

In some embodiments, the kit further comprises at least one second nucleic acid molecule encoding: Cas9 protein, an RFP, or both.

In some embodiments, the kit comprises a first nucleic acid molecule encoding a gRNA as disclosed herein, a second nucleic acid molecule encoding a Cas9 protein, and a third nucleic acid molecule encoding an RFP.

In some embodiments, the nucleic acid molecules as disclosed herein, are integrated in or reside in at least one expression vector or a plasmid.

In some embodiments, the first nucleic acid molecule and the second nucleic acid molecule are integrated in or reside in the same expression vector or a plasmid. In some embodiments, the first nucleic acid molecule and the second nucleic acid molecule are integrated in or reside in a first expression vector or a plasmid and the third nucleic acid molecule is integrated in or resides in a second expression vector or a plasmid.

In some the kit comprises: (i) at least one first nucleic acid molecule encoding a gRNA, comprising the nucleotide sequence as set forth in any one of SEQ ID Nos: 1-3, and 18-20, (ii) a second nucleic acid molecule comprising a sequence encoding Cas9 protein; and (iii) a third nucleic acid molecule comprising a sequence encoding RFP, as disclosed herein.

In some embodiments, the second nucleic acid molecule encoding Cas9 protein comprises the nucleotide sequence: ATGGCCCCAAAGAAGAAGCGGAAGGTCGGTATCCACGGAGTCCCAGCAGCCGACA AGAAGTACAGCATCGGCCTGGACATCGGCACCAACTCTGTGGGCTGGGCCGTGAT CACCGACGAGTACAAGGTGCCCAGCAAGAAATTCAAGGTGCTGGGCAACACCGAC CGGCACAGCATCAAGAAGAACCTGATCGGAGCCCTGCTGTTCGACAGCGGCGAAA CAGCCGAGGCCACCCGGCTGAAGAGAACCGCCAGAAGAAGATACACCAGACGGA AGAACCGGATCTGCTATCTGCAAGAGATCTTCAGCAACGAGATGGCCAAGGTGGA CGACAGCTTCTTCCACAGACTGGAAGAGTCCTTCCTGGTGGAAGAGGATAAGAAG CACGAGCGGCACCCCATCTTCGGCAACATCGTGGACGAGGTGGCCTACCACGAGA AGTACCCCACCATCTACCACCTGAGAAAGAAACTGGTGGACAGCACCGACAAGGC CGACCTGCGGCTGATCTATCTGGCCCTGGCCCACATGATCAAGTTCCGGGGCCACT TCCTGATCGAGGGCGACCTGAACCCCGACAACAGCGACGTGGACAAGCTGTTCAT CCAGCTGGTGCAGACCTACAACCAGCTGTTCGAGGAAAACCCCATCAACGCCAGC GGCGTGGACGCCAAGGCCATCCTGTCTGCCAGACTGAGCAAGAGCAGACGGCTGG AAAATCTGATCGCCCAGCTGCCCGGCGAGAAGAAGAATGGCCTGTTCGGAAACCT GATTGCCCTGAGCCTGGGCCTGACCCCCAACTTCAAGAGCAACTTCGACCTGGCCG AGGATGCCAAACTGCAGCTGAGCAAGGACACCTACGACGACGACCTGGACAACCT GCTGGCCCAGATCGGCGACCAGTACGCCGACCTGTTTCTGGCCGCCAAGAACCTGT CCGACGCCATCCTGCTGAGCGACATCCTGAGAGTGAACACCGAGATCACCAAGGC CCCCCTGAGCGCCTCTATGATCAAGAGATACGACGAGCACCACCAGGACCTGACC CTGCTGAAAGCTCTCGTGCGGCAGCAGCTGCCTGAGAAGTACAAAGAGATTTTCTT CGACCAGAGCAAGAACGGCTACGCCGGCTACATTGACGGCGGAGCCAGCCAGGAA GAGTTCTACAAGTTCATCAAGCCCATCCTGGAAAAGATGGACGGCACCGAGGAAC TGCTCGTGAAGCTGAACAGAGAGGACCTGCTGCGGAAGCAGCGGACCTTCGACAA CGGCAGCATCCCCCACCAGATCCACCTGGGAGAGCTGCACGCCATTCTGCGGCGG CAGGAAGATTTTTACCCATTCCTGAAGGACAACCGGGAAAAGATCGAGAAGATCC TGACCTTCCGCATCCCCTACTACGTGGGCCCTCTGGCCAGGGGAAACAGCAGATTC GCCTGGATGACCAGAAAGAGCGAGGAAACCATCACCCCCTGGAACTTCGAGGAAG TGGTGGACAAGGGCGCTTCCGCCCAGAGCTTCATCGAGCGGATGACCAACTTCGAT AAGAACCTGCCCAACGAGAAGGTGCTGCCCAAGCACAGCCTGCTGTACGAGTACT TCACCGTGTATAACGAGCTGACCAAAGTGAAATACGTGACCGAGGGAATGAGAAA GCCCGCCTTCCTGAGCGGCGAGCAGAAAAAGGCCATCGTGGACCTGCTGTTCAAG ACCAACCGGAAAGTGACCGTGAAGCAGCTGAAAGAGGACTACTTCAAGAAAATCG AGTGCTTCGACTCCGTGGAAATCTCCGGCGTGGAAGATCGGTTCAACGCCTCCCTG GGCACATACCACGATCTGCTGAAAATTATCAAGGACAAGGACTTCCTGGACAATG AGGAAAACGAGGACATTCTGGAAGATATCGTGCTGACCCTGACACTGTTTGAGGA CAGAGAGATGATCGAGGAACGGCTGAAAACCTATGCCCACCTGTTCGACGACAAA GTGATGAAGCAGCTGAAGCGGCGGAGATACACCGGCTGGGGCAGGCTGAGCCGG AAGCTGATCAACGGCATCCGGGACAAGCAGTCCGGCAAGACAATCCTGGATTTCC TGAAGTCCGACGGCTTCGCCAACAGAAACTTCATGCAGCTGATCCACGACGACAG CCTGACCTTTAAAGAGGACATCCAGAAAGCCCAGGTGTCCGGCCAGGGCGATAGC CTGCACGAGCACATTGCCAATCTGGCCGGCAGCCCCGCCATTAAGAAGGGCATCCT GCAGACAGTGAAGGTGGTGGACGAGCTCGTGAAAGTGATGGGCCGGCACAAGCCC GAGAACATCGTGATCGAAATGGCCAGAGAGAACCAGACCACCCAGAAGGGACAG AAGAACAGCCGCGAGAGAATGAAGCGGATCGAAGAGGGCATCAAAGAGCTGGGC AGCCAGATCCTGAAAGAACACCCCGTGGAAAACACCCAGCTGCAGAACGAGAAGC TGTACCTGTACTACCTGCAGAATGGGCGGGATATGTACGTGGACCAGGAACTGGA CATCAACCGGCTGTCCGACTACGATGTGGACCATATCGTGCCTCAGAGCTTTCTGA AGGACGACTCCATCGACAACAAGGTGCTGACCAGAAGCGACAAGAACCGGGGCA AGAGCGACAACGTGCCCTCCGAAGAGGTCGTGAAGAAGATGAAGAACTACTGGCG GCAGCTGCTGAACGCCAAGCTGATTACCCAGAGAAAGTTCGACAATCTGACCAAG GCCGAGAGAGGCGGCCTGAGCGAACTGGATAAGGCCGGCTTCATCAAGAGACAGC TGGTGGAAACCCGGCAGATCACAAAGCACGTGGCACAGATCCTGGACTCCCGGAT GAACACTAAGTACGACGAGAATGACAAGCTGATCCGGGAAGTGAAAGTGATCACC CTGAAGTCCAAGCTGGTGTCCGATTTCCGGAAGGATTTCCAGTTTTACAAAGTGCG CGAGATCAACAACTACCACCACGCCCACGACGCCTACCTGAACGCCGTCGTGGGA ACCGCCCTGATCAAAAAGTACCCTAAGCTGGAAAGCGAGTTCGTGTACGGCGACT ACAAGGTGTACGACGTGCGGAAGATGATCGCCAAGAGCGAGCAGGAAATCGGCA AGGCTACCGCCAAGTACTTCTTCTACAGCAACATCATGAACTTTTTCAAGACCGAG ATTACCCTGGCCAACGGCGAGATCCGGAAGCGGCCTCTGATCGAGACAAACGGCG AAACCGGGGAGATCGTGTGGGATAAGGGCCGGGATTTTGCCACCGTGCGGAAAGT GCTGAGCATGCCCCAAGTGAATATCGTGAAAAAGACCGAGGTGCAGACAGGCGGC TTCAGCAAAGAGTCTATCCTGCCCAAGAGGAACAGCGATAAGCTGATCGCCAGAA AGAAGGACTGGGACCCTAAGAAGTACGGCGGCTTCGACAGCCCCACCGTGGCCTA TTCTGTGCTGGTGGTGGCCAAAGTGGAAAAGGGCAAGTCCAAGAAACTGAAGAGT GTGAAAGAGCTGCTGGGGATCACCATCATGGAAAGAAGCAGCTTCGAGAAGAATC CCATCGACTTTCTGGAAGCCAAGGGCTACAAAGAAGTGAAAAAGGACCTGATCAT CAAGCTGCCTAAGTACTCCCTGTTCGAGCTGGAAAACGGCCGGAAGAGAATGCTG GCCTCTGCCGGCGAACTGCAGAAGGGAAACGAACTGGCCCTGCCCTCCAAATATG TGAACTTCCTGTACCTGGCCAGCCACTATGAGAAGCTGAAGGGCTCCCCCGAGGAT AATGAGCAGAAACAGCTGTTTGTGGAACAGCACAAGCACTACCTGGACGAGATCA TCGAGCAGATCAGCGAGTTCTCCAAGAGAGTGATCCTGGCCGACGCTAATCTGGA CAAAGTGCTGTCCGCCTACAACAAGCACCGGGATAAGCCCATCAGAGAGCAGGCC GAGAATATCATCCACCTGTTTACCCTGACCAATCTGGGAGCCCCTGCCGCCTTCAA GTACTTTGACACCACCATCGACCGGAAGAGGTACACCAGCACCAAAGAGGTGCTG GACGCCACCCTGATCCACCAGAGCATCACCGGCCTGTACGAGACACGGATCGACC TGTCTCAGCTGGGAGGCGACAAAAGGCCGGCGGCCACGAAAAAGGCCGGCCAGG CAAAAAAGAAAAAGTAA (SEQ ID NO: 9), or an analog thereof, having at least 50%, 60%, 70%, 80%, 90%, 95%, 99% identity or homology thereto, or any value and range therebetween. Each possibility represents a separate embodiment of the invention. In some embodiments, an analog of the second nucleic acid molecule encoding Cas9, comprises 50-100%, 60-100%, 70%-100%, 80%-100%, 90%-100%, 95%-100%, 99%-100% identity or homology to a nucleic acid sequence as set forth in SEQ ID NO: 9. Each possibility represents a separate embodiment of the invention.

In some embodiments, the third nucleic acid molecule encoding RFP comprises a nucleic acid sequence as set forth in SEQ ID NO: 8, or an analog thereof as disclosed herein.

In some embodiments, the third nucleic acid molecule comprises a promoter. In some embodiments, the promoter comprises and a chicken β-actin (CBA) promoter. In some embodiments, the promoter comprises a CBh promoter. In some embodiments, the promotor comprises the nucleotide sequence:

(SEQ ID NO: 10)
GAATTCCGTTACATAACTTACGGTAAATGGCCCGCCTGGCTGACCGCCC
AACGACCCCCGCCCATTGACGTCAATAGTAACGCCAATAGGGACTTTCC
ATTGACGTCAATGGGTGGAGTATTTACGGTAAACTGCCCACTTGGCAGT
ACATCAAGTGTATCATATGCCAAGTACGCCCCCTATTGACGTCAATGAC
GGTAAATGGCCCGCCTGGCATTGTGCCCAGTACATGACCTTATGGGACT
TTCCTACTTGGCAGTACATCTACGTATTAGTCATCGCTATTACCATGGT
CGAGGTGAGCCCCACGTTCTGCTTCACTCTCCCCATCTCCCCCCCCTCC
CCACCCCCAATTTTGTATTTATTTATTTTTTAATTATTTTGTGCAGCGA
TGGGGGCGGGGGGGGGGGGGGGGCGCGCGCCAGGCGGGGCGGGGCGGGG
CGAGGGGCGGGGGGGGCGAGGCGGAGAGGTGCGGCGGCAGCCAATCAGA
GCGGCGCGCTCCGAAAGTTTCCTTTTATGGCGAGGCGGCGGCGGCGGCG
GCCCTATAAAAAGCGAAGCGCGCGGCGGGCGGGAGTCGCTGCGCGCTGC
CTTCGCCCCGTGCCCCGCTCCGCCGCCGCCTCGCGCCGCCCGCCCCGGC
TCTGACTGACCGCGTTACTCCCACAGGTGAGCGGGCGGGACGGCCCTTC
TCCTCCGGGCTGTAATTAGCTGAGCAAGAGGTAAGGGTTTAAGGGATGG
TTGGTTGGTGGGGTATTAATGTTTAATTACCTGGAGCACCTGCCTGAAA
TCACTTTTTTTCAGGTTGGCGCCACC.

In some embodiments, the third nucleic acid molecule comprises a nucleotide sequence encoding a polyadenylation (polyA) signal. In some embodiments, a nucleotide sequence encoding a polyA signal comprises the nucleotide sequence:

(SEQ ID NO: 11)
CGGCCGCGACTCTAGATCATAATCAGCCATACCACATTTGTAGAGGTTT
TACTTGCTTTAAAAAACCTCCCACACCTCCCCCTGAACCTGAAACATAA
AATGAATGCAATTGTTGTTGTTAACTTGTTTATTGCAGCTTATAATGGT
TACAAATAAAGCAATAGCATCACAAATTTCACAAATAAAGCATTTTTTT
CACTGCATTCTAGTTGTGGTTTGTCCAAACTCATCAATGTATCTTAAGG
CGTGGATCC.

In some embodiments, the third nucleic acid molecule comprises a nucleotide sequence that enables integration of the exogenous reporter gene into the at least one location of a Z gender chromosome. In some embodiments, SEQ ID NO: 8, or analog thereof, is flanked at 5′ thereof, 3′ thereof, and both, by a homologous arm. In some embodiments, integration of the exogenous reporter gene into the at the least one location of a Z gender chromosome is by homology directed repair (HDR) of a homologues arm to a sequence within the genomic site disclosed herein.

The term “homology-directed repair (HDR)”, as used herein encompasses a process where a DNA double-strand break (DSB) is repaired by homologous recombination using a DNA template. The term “homologous arm”, as used herein refers to an HDR template introduced into a specific vector or plasmid, designed to align a functional gene to a specific location in the genome. In some embodiments, where CRISPR is used as a PEN, the arms sequences (e.g., 5′ homology arm; left, upstream, and 3′ homology arm; right, downstream) comprise between about 10 to 5,000 bp, between about 50 to 1,000 bp, or between about 100 to 500 bp.

In some embodiments, the third nucleic acid molecule comprises a 5′ homology arm. In some embodiments, the 5′ homology arm comprises the nucleotide sequence:

(SEQ ID NO: 12)
GGCCTATACAGACAAATCTTTACTAAATGGAGAATCCTGACATTTTGTC
CATTTCTCTCTATACCACCAACTCTCATCTTGGTTACATTGCCTGTCAG
TTGAACAGGCAAGATCTCCATTCCCAAAACACCACACTTATCTTGAATT
GAGCCATCAAGAGGTATTTCTCTGATACACCATTATCTAAAACCCAGTG
TTATTTTTCAGTGAGGGTAGATTCCCTTTACTGCTTTTTCTCCTCATTT
CCA.

In some embodiments, the third nucleic acid molecule comprises a 3′ homology arm. In some embodiments, the 3′ homology arm comprises the nucleotide sequence:

(SEQ ID NO: 13)
ATAACGGCATTCAGTACTTGTCATCACTAATTAGTGTATTCAATCTACC
CTCTATTTCTAGGACAACATGTTTTAATAATTCACCATAAATTAGTTCT
ATGAGCATTTTGATCTAGTGGTTGTTGTAGATCACAGGATATGAATAGA
TTGTGTGTATGTGTAAACCAGGAGTGTCTTGACAAAAGGACACAGTGCT
GGTAAGACCAGCAGAAGAGGGTCCCCTGTGGCAAAAATGTTTTACTGAC
CTTTG.

In some embodiments, a third nucleic acid molecule comprising a nucleotide sequence as set forth in: SEQ ID NO: 12, SEQ ID NO: 13, and both, is used to integrate the exogenous reporter gene into site 4a.

In some embodiments, the 5′ homology arm comprises the nucleotide sequence:

(SEQ ID NO: 14)
AATACACAGCACTGTATCTTTTATGGCAATCTATTTTCGATTGTCTCAT
GTGCAGAACAGTTACCAGGATTGCAACAACGGATTGAATTTATCCGAAA
AGAAGTTTTGTGTCCTGCTTTGTGATAGCTGAGAAAGAAAGGCAGTGAT
GCTTAAAAAGCAGTCAGTGACCTAATCACCTACTGTCAGGTGTACTATG
AATACATACAGTAGAGCCAGTAACACAGTTTGACAGCATTTTCATTAGA
TGTTT.

In some embodiments, the 3′ homology arm comprises the nucleotide sequence:

(SEQ ID NO: 15)
CAGAACAAAATAGTATTTTTGTTCACAACTGGGAGTGAAATCTGATTTC
AAACCACTAAAAAGAATAGTGGAGACATGAAGAAAAAACGTTTTGTCTG
AATGCTTTCTTGGGTAGTCAGAAATAAAAGCTGTTGTACGGAAGATCAT
ATGAGGCTGCTATGGGTAGCAGCATCAAGTGTGGCAGTGGAGCAGAGAG
AGATTGCATGCCACGGGGAGAGGAAATGTGGAAAATTACACATATCACC
GTGAG.

In some embodiments, a third nucleic acid molecule comprising a nucleotide sequence as set forth in: SEQ ID NO: 14, SEQ ID NO: 15, and both, is used to integrate the exogenous reporter gene into site 5a.

In some embodiments, the 5′ homology arm comprises the nucleotide sequence:

(SEQ ID NO: 16)
GGTCTGTGCAAACAGTGTTTCTCATGCAACTTGGCTGCCTTTAAATGGC
TTACCAACTCTTTTCTGAAAAACCTAAAAAATGTCTGTGTGCCAAGTAA
GATACTTCAATTCAAAAGGAGGTTTTTCAATTTTTTCTCTCAGTTTATC
ATTTCTTCTACTTGAAAAATATATTTTAATTTTAATGTTTTTTGTTTTA
TACAAAAATATATGAACTATGTATTACTATTGTCACCTGTCATTACCAA
GAAGT.

In some embodiments, the 3′ homology arm comprises the nucleotide sequence:

(SEQ ID NO: 17)
ACATCACCAGGGCTGGAGGTTACATCCTGCTGGCCAAAACTACATTATG
GTGGGTTAGCTAATTTTACAGGAGGGATAAATTGTGAGAATCTGGAAAC
CATAATCTTGACAAGAAAAAATTAACACCCAATTCCTTGGTGAGACTGG
GCATTATATGGACATGGGAAATCTCACAGTCATGATATATGTTAGGAAG
GAACTCTCCTTCAAGGGTCCAGGACTGTAAGTATTGGCCTGCCCCAACA
CTTGG.

In some embodiments, a third nucleic acid molecule comprising a nucleotide sequence as set forth in: SEQ ID NO: 16, SEQ ID NO: 17, and both, is used to integrate the exogenous reporter gene into site 14a.

In some embodiments, the 5′ homology arm comprises the nucleotide sequence:

(SEQ ID NO: 21)
TGAATGTGGAAAGTGAAGATAACTACTAGTGCAGCAAGCAGACATAGAG
ATTTTATAAAAGTGGATGGTGAGCATTGTTTGTTAGATACAAACCATTA
AGCAGAGATGCCTAGTGACTCAGAGTACAGAGCTTCCTGACTGTCAACA
CTGACCATGACACTGACCACGTTATGATCCAGGAATAATAGAACCTGTA
TTTACTCTTAGATATTCTTAAATTGTAATCACAGAAGAGGACAAACAGT
GATAG.

In some embodiments, the 3′ homology arm comprises the nucleotide sequence:

(SEQ ID NO: 22)
TCTATTGTCTCATAAGTCCTACCACTCAGGAATAAGGCTTATAAGAAAC
ATGAGAAATGTGTAACTGTAGAGTGAGTTAGACATAGCCAGCAGCTATA
TACGTACCCATATGCTCACAGTCTCTCTCCTTCGCAGGATTAGGAAATA
AAATTGGGTGAAAAAGGTTATAGATAAGACAGAGATAGAAAGATTGCTA
ATCATTTAACATATTTGATTTGGGCAAAGTAATTGAATACACTGTCAAG
TAAAG.

In some embodiments, a third nucleic acid molecule comprising a nucleotide sequence as set forth in: SEQ ID NO: 21, SEQ ID NO: 22, and both, is used to integrate the exogenous reporter gene into site 7b.

In some embodiments, the 5′ homology arm comprises the nucleotide sequence:

(SEQ ID NO: 23)
CAAGAACATTAAAAAGAAACAGGATCTCTCTTGCTTTTGTTAAGAAAAA
AAAACAGCAGCAGTACACCAAGAGCAGTTAAATTACTCTATTTAGAGAT
GTCTGAAACACTTCTAAAGCAAAGCAGGTAATACTTCAGGGACCATCTA
CAAGCTTGCAGTTTTAGCTCCCTTCTATAACTTTTCCAATTTGATCTTT
CTGTGGAAAAATACAAAATTGTGTCTGTTTCTTGAAGACCGGTAACCAG
AAAGT.

In some embodiments, the 3′ homology arm comprises the nucleotide sequence:

(SEQ ID NO: 24)
TATTTGTCAGAAAAATCTATCTGCATACTCATTTCTTTGAATGAGATTA
CAATCATGAGATGTCCACTATCTGCATTTTTGATCACTAGTGAAAACTC
CTTCTTTCCAAAGCCACTGGTGATTATTGTTTATTTAGGAGAGCAGGCC
TTTAAAAGAAATACTGTGGTCACCTGTGTGACTGAAAAAAGCACATATT
CAATTCATGTGAATAACATTAGAAAGTTTCCAGGAACTGCTGATGATTC
AAGCA.

In some embodiments, a third nucleic acid molecule comprising a nucleotide sequence as set forth in: SEQ ID NO: 23, SEQ ID NO: 24, and both, is used to integrate the exogenous reporter gene into site 8a.

In some embodiments, the 5′ homology arm comprises the nucleotide sequence:

(SEQ ID NO: 25)
GAAACATCATTTATAGACTAAGTGTGTATATTATAAATCTGCTGATTGC
ATAGGTAAATTTTTTTCAGTATCATCTGCTGTCAAAATTTTGGCTGTGA
TGAAATCAACTTTCTTCATAGAAACTCATATAATGCTGTGCTTCGGATT
TTTGATGAAAATAGTGATGATAATACACCAGTGTTCCAGTTGTGACAGA
GCAGTGCTTACAGAGTCAAAAACTTTTTATTTTATTGTGCTATCCTGCC
AATGA.

In some embodiments, the 3′ homology arm comprises the nucleotide sequence:

(SEQ ID NO: 26)
TGCTTTGCTTGCACATGCAGTAAACTTTTTTAATCTTAATTCATAAACT
CTCACACTTTTACATTTCCAATTTTCACATCTTTAATCATCTGGGAGAT
GATTTCACATCCTCATCAAGTTTGTGGATGACCCAGAACTGAGGGCAGT
GGCTAACTCACCAGAGTGCTGTGCTGCCATCTGGAGGAACTTGCACAGT
TGGAGAAGGGCTGACAGGAACTTGATGGAGTTCTACATGGAGAAGGGAA
AGTTC.

In some embodiments, a third nucleic acid molecule comprising a nucleotide sequence as set forth in: SEQ ID NO: 25, SEQ ID NO: 26, and both, is used to integrate the exogenous reporter gene into site 13a.

In some embodiments, the at least one first nucleic acid molecule and any one of: the second nucleic acid molecule and the third nucleic acid molecule, are operably linked. In some embodiments, the at least one first nucleic acid molecule, the second nucleic acid molecule, and the third nucleic acid molecule disclosed herein are operably linked. In some embodiments, the at least one first nucleic acid molecule and the second nucleic acid molecule are operably linked.

The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the kit further comprises instructions for integrating the at least second nucleic acid molecule encoding RFP into at least one location of Z gender chromosome of a female chicken, wherein the at least one location is listed under Table 1. In some embodiments, the at least one location is listed under Table 3. In some embodiments, the at least one location is selected from: site 4a, site 5a, site 13a, site 14a, or any combination thereof.

The term “encoding” is intended to mean that the subject nucleic acid may be transcribed and translated into either the desired polypeptide or the subject protein in an appropriate expression system, e.g., when the subject nucleic acid is linked to appropriate control sequences such as promoter and enhancer elements in a suitable vector (e.g., an expression vector) and when the vector is introduced into an appropriate system or cell. The term “nucleic acid” is intended to mean natural and/or synthetic linear, circular and sequential arrays of nucleotides and nucleosides, e.g., cDNA, genomic DNA (gDNA), mRNA, and RNA, oligonucleotides, oligonucleosides, and derivatives thereof.

It should be appreciated that in some embodiments, at least one of the first and the second nucleic acid sequences provided and used by the methods and kits of the invention may be constructed and comprised within a vector. “Vectors” as used herein, encompass vectors such as plasmids, phagemides, viruses, integratable DNA fragments, and other vehicles, which enable the integration of DNA fragments into the genome of the host, or alternatively, enable expression of genetic elements that are not integrated. Vectors are typically self-replicating DNA or RNA constructs containing the desired nucleic acid sequences, and operably linked genetic control elements that are recognized in a suitable host cell and effect the translation of the desired spacers. Generally, the genetic control elements can include a prokaryotic promoter system or a eukaryotic promoter expression control system. Such system typically includes a transcriptional promoter, transcription enhancers to elevate the level of RNA expression. Vectors usually contain an origin of replication that allows the vector to replicate independently of the host cell. In yet some alternative embodiments, the expression vectors used by the invention may comprise elements necessary for integration of the desired exogenous reporter gene of into the chicken gender specific chromosome Z.

Accordingly, the term “control and regulatory elements” includes promoters, terminators, and other expression control elements. Such regulatory elements are described in Goeddel; [Goeddel., et al., Gene Expression Technology: Methods in Enzymology 185, Academic Press, San Diego, Calif. (1990)]. For instance, any of a wide variety of expression control sequences that control the expression of a DNA sequence when operatively linked to it may be used in these vectors to express DNA sequences encoding any desired protein using the method of this invention.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. The promoter may extend upstream or downstream of the transcriptional start site and may be any size ranging from a few base pairs to several kilo-bases. In some embodiments, the promoter is a promoter of a chicken cell. In some embodiments, the promoter present in Z chromosome of a chicken cell. In some embodiments, the promoter in an inducible promoter.

As used herein, the term “an inducible promoter” encompasses a promoter that is activated only when upon a specific stimulus. Once activated, the inducible promoter binds to RNA polymerase and transcriptional factors, enabling the transcription process.

A vector may additionally include appropriate restriction sites, antibiotic resistance, or other markers for selection of vector-containing cells. Plasmids are the most commonly used form of vector but other forms of vectors which serve an equivalent function, and which are, or become, known in the art are suitable for use herein. See, e.g., Pouwels et al., Cloning Vectors: a Laboratory Manual (1985 and supplements), Elsevier, N.Y.; and Rodriquez, et al. (eds.) Vectors: a Survey of Molecular Cloning Vectors and their Uses, Buttersworth, Boston, Mass (1988), which are incorporated herein by reference.

In some embodiments, the vector is introduced into the cell by standard methods including electroporation (e.g., as described in From et al., Proc. Natl. Acad. Sci. USA 82, 5824 (1985)), heat shock, infection by viral vectors, high velocity ballistic penetration by small particles with the nucleic acid either within the matrix of small beads or particles, or on the surface (Klein et al., Nature 327. 70-73 (1987)), such as biolistic use of coated particles, and needle-like particles, Agrobacterium Ti plasmids and/or the like.

In some embodiments, the nucleic acid molecule is transcribed by RNA polymerase II (RNAP II and Pol II). RNAP II is an enzyme found in eukaryotic cells, known to catalyze the transcription of DNA to synthesize precursors of mRNA and most snRNA and microRNA.

It will be appreciated that other than containing the necessary elements for the transcription and translation of the inserted coding sequence (encoding the polypeptides disclosed herein), the expression construct of the present invention can also include sequences engineered to optimize stability, production, purification, yield, or activity of the expressed polypeptide.

Any concentration ranges, percentage range, or ratio range recited herein are to be understood to include concentrations, percentages, or ratios of any integer within that range and fractions thereof, such as one tenth and one hundredth of an integer, unless otherwise indicated.

Any number range recited herein relating to any physical feature, such as polynucleotides and polypeptides, size, weight, or length, are to be understood to include any integer within the recited range, unless otherwise indicated.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one”, are used interchangeably in this application.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

All publications, patents and patent applications mentioned in this specification are herein incorporated in their entirety by references into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

### EXAMPLES

Generally, the nomenclature used herein, and the laboratory procedures utilized in the present invention include molecular, biochemical, bioengineering, bioprocessing, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); Molecular Cell Biology Berk A. et al. 8th edition; Molecular Biotechnology: Principles and Applications of Recombinant DN, Glick BR. 5th edition; Culture of Animal Cells: A Manual of Basic Technique and Specialized Applications Freshney IR, 7th edition; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference. Other general references are provided throughout this document.

### Example 1

### Bioinformatic Analysis for Identifying New Genomic Safe Harbor Sites

In order to identify genomic locations in chicken Z chromosome which can be targeted with a bioreporter expression cassette, such as a gene encoding the red fluorescent protein, the inventors performed a comprehensive bioinformatic analysis, by applying firm criteria for allocating new potentially genomic safe harbor sites.

The inventors based their bioinformatic analysis on criteria used in previously published work that aimed to identify genomic safe harbor sites on other genomes. The differences in the size of the genome and the number of chromosomes were taken into consideration in the analysis (e.g., 39 chromosomes containing ~1.2 Gb for the chicken genome, compared to 20 chromosomes containing ~2.5 Gb and 23 chromosomes containing ~3 Gb in the mouse and human genomes, respectively). The following criteria were set for identifying safe harbor genomic sites:
Criteria 1—the distance of the genomic site from the end of any gene should be greater than 20 kb.
Criteria 2—the distance of the genomic site from any cancer-related gene should be greater than 120 kb.
Criteria 3—the distance of the genomic site from any microRNA (miRNA) should be greater than 120 kb.
Criteria 4—the genomic site should be located outside CpG islands, which generally represent transcription units such as topologically associating domain(s) TAD(s), enhancers, or promoters.
Criteria 5—the genomic site should be located outside ultraconserved genomic regions.
Criteria 6—the genomic site should exclude repeated elements.

The inventors performed the bioinformatic analysis using the platform offered by the University of California, Santa Cruz (UCSC) genome browser.

### Genomic Data, Assembly, Mapping, and Sequencing:

UCSC Genome Browser assembly ID: galGal6
Sequencing/Assembly provider ID: Genome Reference Consortium GRCg6a
Assembly date: March 2018
Accession ID: GCF_000002315.5
NCBI Genome ID: 111 (Gallus gallus) #NCBI Assembly ID: 1668981®
NCBI BioProject ID: 13342
NCBI BioSample ID: SAMN02981218

### Genes and Genes Predictions:

TransMap Alignments Version 5 tracks, (Data last updated at UCSC: 2019 Jun. 10)
TransMap Ensembl and GENCODE Mappings Version 5
Ensembl Gene Predictions, Source data version: 104, (Data last updated at UCSC: 2021-05-25)
RefSeq gene predictions from NCBI-Annotation Release NCBI Gallus gallus Annotation Release 104, (Data last updated at UCSC: 2020-03-29)
Non-Chicken RefSeq Genes
Chicken mRNAs from GenBank
Non-Chicken mRNAs from GenBank®
UniProt SwissProt/TrEMBL Protein Annotations, (Source data version at UCSC: UniProt Knowledgebase Release 2020_05)
   miRNA Predictions:
miRBase
MiRscan

### Expression and Regulation:

CpG Islands tracks (Islands <300 bp)

### Comparative Genomics:

Vertebrate Multiz Alignment & Conservation (multiple alignments of all 77 vertebrate species)
Birds Chain and Net Alignments

### Variation and Repeats:

Repeating Elements by RepeatMasker (Short interspersed nuclear elements, including ALUs, Long interspersed nuclear elements, Long terminal repeat elements including retroposons, Satellites, micro-satellites, Low complexity repeats, RNA repeats).

The inventors successfully identified 18 potential genomic locations in the Z gender chromosome, by their unbiased bioinformatic screen, which met the stringent criteria that were applied.

TABLE 1
Initial screen of safe harbor loci on the Z gender chromosome
by applying criteria Nos: 1-6. Sites are in accordance with
or based on the UCSC Genome Browser assembly ID: galGal6.

| | Genomic location (nucleotide number on Z gender |
|---|---|
| Location No. | chromosome) |
| Site 1 | chrZ: 1,550,000-1,730,000 |
| Site 2 | chrZ: 4,075,000-6,150,000 |
| Site 3 | chrZ: 17,100,000-17,450,000 |
| Site 4 | chrZ: 17,550,000-18,050,000 |
| Site 5 | chrZ: 19,500,000-20,000,000 |
| Site 6 | chrZ: 26,000,000-26,400,000 |
| Site 7 | chrZ: 30,000,000-30,800,000 |
| Site 8 | chrZ: 36,000,000-36,600,000 |
| Site 9 | chrZ: 38,500,000-39,200,000 |
| Site 10 | chrZ: 48,300,000-50,000,000 |
| Site 11 | chrZ: 50,900,000-51,400,000 |
| Site 12 | chrZ: 51,425,000-51,625,000 |
| Site 13 | chrZ: 58,500,000-59,700,000 |
| Site 14 | chrZ: 61,400,000-62,400,000 |
| Site 15 | chrZ: 63,150,000-63,550,000 |
| Site 16 | chrZ: 64,350,000-65,250,000 |
| Site 17 | chrZ: 69,700,000-70,400,000 |
| Site 18 | chrZ: 73,500,000-78,800,000 |

TABLE 2
Size and additional characteristics of genomic sites Nos: 1-18

| | | | | miRNA | |
|---|---|---|---|---|---|
| Location | | | | (Y or | |
| No. | Size | | Comments | N) | comments |
| Site 1 | 165 | Kb | 2 human mirRNAs, | N | |
| | | | neighboring with | | |
| | | | Smad2 | | |
| Site 2 | 2 | Mb | human mirRNAs, | N | |
| | | | several loci | | |
| Site 3 | 260 | Kb | One human | N | |
| | | | lncRNA, one mouse | | |
| | | | gm | | |
| Site 4 | 460 | Kb | Few very small loci | N | |
| Site 5 | 420 | Kb | OK | N | Closest mirRNA: |
| | | | | | 18874996 |
| | | | | | (875 Kb) |
| Site 6 | 290 | Kb | Several loci | N | Closest mirRNA: |
| | | | | | 27510654 (1 Mb) |
| Site 7 | 700 | Kb | One small gene in | N | |
| | | | the middle | | |
| Site 8 | 480 | Kb | Few very small loci | N | Closest mirRNA: |
| | | | | | 35181264 |
| | | | | | (800 Kb) |
| Site 9 | 600 | Kb | Several loci, in | N | Closest mirRNA: |
| | | | proximity to the | | 40078117 |
| | | | Centromere | | (800 Kb) |
| Site 10 | 1.5 | Mb | One human gene in | Y | mirRNA at |
| | | | the middle | | 48684570 |
| Site 11 | 450 | Kb | Several Meleag | N | |
| | | | loci, near CHD1Z | | |
| | | | gene, | | |
| Site 12 | 185 | Kb | One long Meleag | N | |
| | | | locus | | |
| Site 13 | 800 | Kb | Multiple Ensemble | N | Closest mirRNA: |
| | | | and predictions | | 60469242 |
| | | | loci. | | (900 Kb) |
| Site 14 | 900 | Kb | Two Cuteron loci | N | 2 close mirRNAs: |
| | | | on the side. Two | | (500 Kb) and |
| | | | small loci in the | | (450 Kb) |
| | | | middle. | | |
| Site 15 | 350 | Kb | One human locus in | N | Closest mirRNA: |
| | | | the middle | | 64304208 |
| | | | | | (900 Kb) |
| Site 16 | 800 | Kb | Multiple Ensembke | Y | mirRNA at |
| | | | and small loci | | 64670256; |
| | | | | | mirRNA close |
| | | | | | (40 Kb); |
| Site 17 | 700 | Kb | Some Cuteron loci, | N | |
| | | | close to Jae Han | | |
| | | | insertion site | | |
| Site 18 | 5.1 | Mb | Multiple Ensemble | N | In proximity to |
| | | | and LOC | | mirRNA at |
| | | | predictions | | 78512252 |

Next, the inventors filtered out from genomic sites Nos: 1-18 the genomic locations which did not meet the following cytogenetics-driven criteria:
Criteria 7—sites should be located outside the Z gender chromosome's centromere (~chrZ: 42,150,000-42,260,000).
Criteria 8—sites should be located outside ‘ZW asynapsis’, the locations with a potential risk of recombination between the Z and the W gender chromosomes.

Following subjecting genomic sites Nos: 1-18 to criteria Nos: 7 and 8, the inventors narrowed their list down to 10 locations on the Z gender chromosome, as described in Table 3. First, of the 18 origin potential sites, only genomic locations within sites Nos: 3, 4, 5, 7, 8, 13, 14, and 15, were found to meet the cytogenetics-driven criteria. Moreover, by applying these cytogenetics-driven criteria, the size of each genomic site that met these criteria was narrowed. The new narrowed safe harbor loci, that were found to meet criteria Nos: 1-8, were termed as the origin site number with an addition of a small latter (e.g., site 3a is the narrowed genomic location of site 3). As demonstrated in Table 3, in regard to site Nos: 7 and 14, two potential different safe harbor loci were found for each site, termed as site Nos: 7a, 7b, 14a and 14b, respectively.

TABLE 3
Safe harbor loci on the Z chromosome
post cytogenetic-driven criteria

| | Genomic location (nucleotide |
|---|---|
| Location No. | number on Z chromosome) |
| Site 3a | chrZ: 17,141,000-17,406,000 |
| Site 4a | chrZ: 17,735,000-18,020,000 |
| Site 5a | chrZ: 19,560,000-19,995,000 |
| Site 7a | chrZ: 30,060,000-30,270,000 |
| Site 7b | chrZ: 30,465,000-30,750,000 |
| Site 8a | chrZ: 36,215,000-36,545,000 |
| Site 13a | chrZ: 59,100,000-59,430,000 |
| Site 14a | chrZ: 61,530,000-61,865,000 |
| Site 14b | chrZ: 61,875,000-62,315,000 |
| Site 15a | chrZ: 63,210,000-63,440,000 |

Next, the inventors ranked the ten sites described in Table 3, according to an internal scoring system based on all of their findings. The top-ranked four sites were found to be: site 4a, site 5a, site 7b, and sites 14a, as described in Table 4.

TABLE 4
Top ranked sites on the Z gender chromosome for
insertion of a bioreporter expression cassette

| Location No. | Genomic location | Genomic region size |
|---|---|---|
| Site 4a | chrZ: 17,735,000-18,020,000 | 285 Kb |
| Site 5a | chrZ: 19,560,000-19,995,000 | 435 Kb |
| Site 7b | chrZ: 30,465,000-30,750,000 | 285 Kb |
| Site 14a | chrZ: 61,530,000-61,865,000 | 335 Kb |

Next, the inventors designed multiple gRNAs corresponding to sites Nos: 4a, 5a, 7b, and 14a, to assess their accessibility to Cas9 and potential gene editing and knock-in (KI) in these sites. The gRNAs were designed using the CCTop-CRISPR/Cas9 target online predictor. The gRNAs were designed according to the following parameters:
Cas9: Streptococcus pyogenes Cas9 (a single gRNA per site)
The protospacer adjacent motif (PAM) site: NGG
Genome target: Gallus Gallus, Ensemble V103
Core length=12 bp
Protospacer length=20 bp
Off-target score: CRISPRater score >0.74.
Off-target max. core mismatches=2
Off-target search method: comprehensive and exhaustive across the entire genome.

Among all examined gRNAs, three gRNA molecules, termed gRNA6.1 (SEQ ID NO: 1), gRNA7.2 (SEQ ID NO: 2), and gRNA9.1 (SEQ ID NO: 3), were found to be most suitable for DNA cleavage within sites Nos: 4a, 5a, and 14a, respectively. The sequences of these gRNAs are described in Table 5 hereinbelow. Thus, of the original 18 potential sites, three final genomic sites within the Z gender chromosome were selected, with three corresponding gRNA molecules for further evaluation.

TABLE 5
Top three location sites and their corresponding gRNA molecules.

| | gRNA# | | | |
|---|---|---|---|---|
| | (SEQ ID | | | CRISPRater |
| Location No. | NO:) | gRNA Sequence | Strand | score |
| Site 4a | gRNA6. 1 | ACATGCAATACACTGAACTG | +1 | 0.80 |
| | (SEQ ID | | | |
| | NO: 1) | | | |
| Site 5a | gRNA7.2 | ACTGTTAACAAGGTTGGTTG | +1 | 0.88 |
| | (SEQ ID | | | |
| | NO: 2) | | | |
| Site 14a | gRNA9.1 | CTTGTAGGGCTTGATTACTG | +1 | 0.85 |
| | (SEQ ID | | | |
| | NO: 3) | | | |

The next objective was to evaluate the potential efficiency of gene editing in the above-mentioned three top-ranked sites, with their corresponding gRNA molecules (Table 5). DF1 cells, a cell line of chicken embryo fibroblasts, were separately electroporated with Cas9/guide RNA (gRNA) ribonucleoprotein (RNP) complexes to test DNA cleavage efficiency in the top-ranked sites: site 4a, site 5a, and site 14a, with the corresponding gRNA molecule. DNA samples were tested using the T7 endonuclease I (T7EI) mismatch cleavage assay, which determines on-target genome editing and provides estimation of genome editing efficiency in CRISPR-treated cells.

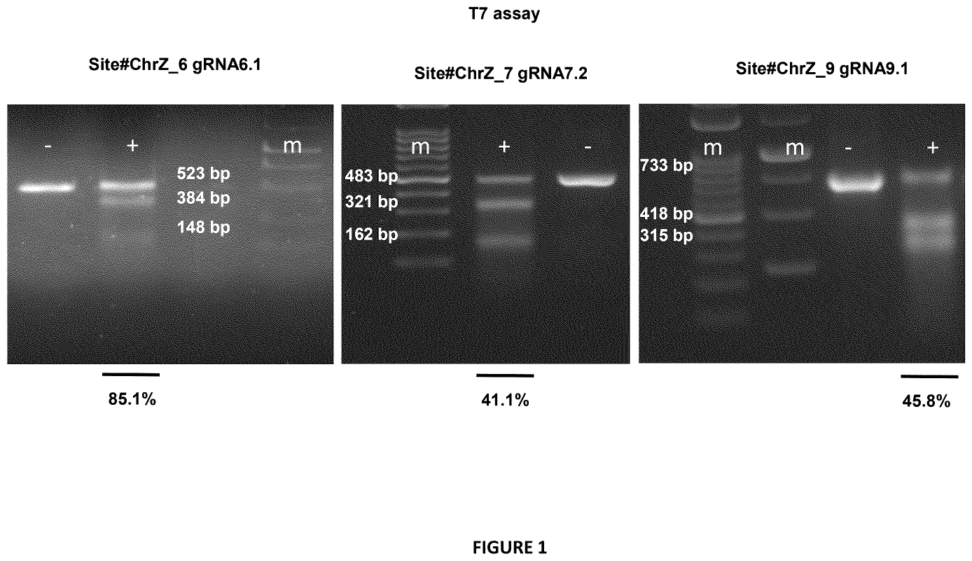

As demonstrated in FIG. 1, according to T7E1 assay, 85.1%, 41.1%, and 45.8% of the dsDNA molecules were cleaved by Cas9/gRNA RNP complexes, comprising gRNA6.1, gRNA7.2, and gRNA9.1, respectively, therefore, indicating a relatively high potential for these loci as appropriate for insertion of the exogenous RFP reporter gene.

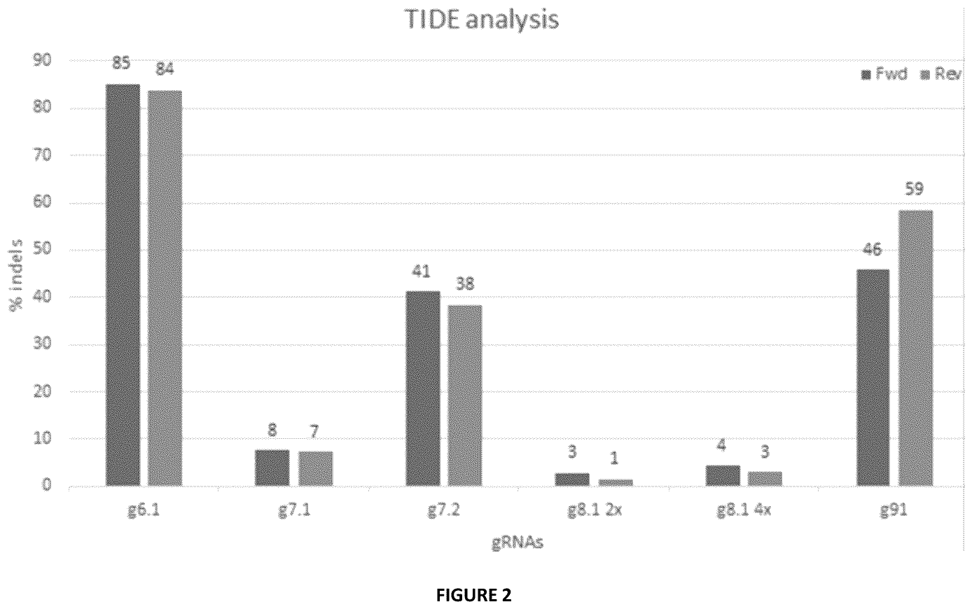

Next, DNA samples were subjected to Sanger sequencing followed by TIDE analyses for tracking DNA insertions and deletions (indels). As seen in FIG. 2, the highest percentage of indels was observed for the examined gRNA molecules: gRNA6.1 (SEQ ID NO: 1), gRNA7.2 (SEQ ID NO: 2), and gRNA9.1 (SEQ ID NO: 3), as compared to other gRNAs molecules candidates.

Therefore, the inventors conclude that following the narrowing process disclosed herein, particular, improved, and non-trivial safe harbor sites for proper integration of a nucleic acid sequence of an exogenous reporter gene encoding RFP into the Z gender chromosome of a chicken have been devised.

### Example 2

### Molecular and Functional Characterization of Genome Edited-Edited Chicken PGCs

To ensure the successful generation of at least three DsRed-positive PGCs, crucial for the subsequent creation of chimeric cockerels and a sex-detectable flock, the inventors focused on five SHL. Selection criteria included the culture viability, proliferation rates, and fluorescence stability. Following careful evaluation, the inventors prioritized three SHL sites that demonstrated the most promising outcomes.

The five SHL initially targeted are specified in Table 6.

TABLE 6

| | Genomic location (nucleotide |
|---|---|
| Location No. | number on Z chromosome) |
| Site 4a | chrZ: 17,735,000-18,020,000 |
| Site 7b | chrZ: 30,465,000-30,750,000 |
| Site 8a | chrZ: 36,215,000-36,545,000 |
| Site 13a | chrZ: 59,100,000-59,430,000 |
| Site 14a | chrZ: 61,530,000-61,865,000 |

Generally, the workflow for each site encompassed five phases: (1) Evaluation of various guide RNAs (gRNAs) targeting the genomic site to facilitate DNA cleavage by Cas9; (2) Construction of two plasmids per site: (i) CRISPR/Cas9 plasmid containing spCas9 and the selected site-specific gRNA, and (ii) DsRed HDR cassette flanked by site-specific homology arms; (3) Introduction of the plasmids into PGC derived from a male Lohmann-LSL chicken embryo; (4) Implementation of sorting procedures to enrich DsRed-positive cells; and (5) Molecular validation to confirm the accurate integration of DsRed into each of the desired SHL.

### Results

Finding Suitable gRNA for Each Targeted Genomic Site

For each of the five genomic SHL, the inventors utilized the CCTop online tool to design gRNAs with the following specifications:
Cas9: Streptococcus pyogenes Cas9 (a single gRNA per site)
The protospacer adjacent motif (PAM) site: NGG
Genome target: Gallus Gallus, Ensemble V103
Core length=12 bp
Protospacer length=20 bp
Off-target score: CRISPRater score >0.74.
Off-target max. core mismatches=2
Off-target search method: comprehensive and exhaustive across the entire genome.

The inventors designed 2-4 gRNAs for each SHL, except for sites 4a and 14a, for which the inventors had previously designed high-quality gRNAs.

Each gRNA underwent testing as a ribonucleoprotein (RNP) complex alongside Cas9 in the chicken DF1 cell line, utilizing electroporation.

DF1 cells were harvested 2-3 days post-electroporation, and DNA was extracted to assess DNA cleavage through a T7 assay.

The T7 products were analyzed on agarose gel, compared to non-transfected and non-cleaved control groups (FIG. 3), and subsequently subjected to sequencing for cleavage quantification via the online TIDE analysis tool.

The gRNAs demonstrating efficient cleavage rates in both gel electrophoresis and TIDE analysis, were selected for further stages (Tables 7-8).

TABLE 7
Quantification of cleavage facilitated by
newly designed gRNAs through TIDE analysis

| | TIDE |
|---|---|
| gRNA | quantification* |
| 7b. 1 | 20% |
| 7b. 2 | 13% |
| 8a. 1 | 12% |
| 8a. 2 | 2% |
| 8a. 3 | 2% |
| 8a. 4 | 6% |
| 13a. 1 | 7% |
| 13a. 2 | 55% |
| 13a. 3 | 45% |
| 13a. 4 | 40% |

*When applicable, average T7 quantification was calculated using both Forward and Reverse sequencing data.

TABLE 8
Selected gRNAs for each SHL based on gel
electrophoresis and TIDE quantification

| | | | SEQ ID |
|---|---|---|---|
| SHL | gRNA | Sequence | NO: |
| 4a | 6.1 | ACATGCAATACACTGAACTG | 1 |
| 7b | 7b.1 | TCTTGCTTTCCACTTTCCAT | 18 |
| 14a | 9.1 | CTTGTAGGGCTTGATTACTG | 3 |
| 8a | 8a.4 | AAGTGTGGAACAAACTGCTG | 19 |
| 13a | 13a.2 | GATGGGCTCCACAAGGAACT | 20 |

### Plasmid Cloning

Based on the selected gRNA sequences, two plasmids were devised for each genomic site

### (1) CRISPR/Cas9 Plasmid

This plasmid harbors spCas9 alongside the site-specific gRNA under a human U6 promoter. Additionally, EGFP serves as a reporter gene to assess transfection efficacy.

Plasmids underwent sequencing to verify the accurate sequences of critical segments, including the Cas9 promoter and ORF, as well as the U6 promoter and gRNA sequence. For sites 4a and 14a, sequencing revealed mutations in these regions. Consequently, the inventors utilized plasmids from other sites, exhibiting correct sequences, for the cloning of gRNA targeting sites 4a and 14a. In these instances, the gRNA promoter was sourced from chicken U6. The resulting cloning products were validated through Sanger sequencing.

### (2) DsRed HDR Plasmid

This plasmid contains the DsRed cassette, comprising the CBh promoter, ORF, and SV40 PolyA signal. The cassette is flanked by 250 bp site-specific homology arms, facilitating integration into the cleaved region of the Z chromosome (Table 9). All cloned plasmids underwent validation via Sanger sequencing.

TABLE 9
Homology arms flanking the DsRed-expressing cassette

| SHL | 5′ Homology arm sequence | 3′ Homology arm sequence |
|---|---|---|
| 4a | GGCCTATACAGACAAATCT | ATAACGGCATTCAGTACTTGTC |
| | TTACTAAATGGAGAATCCT | ATCACTAATTAGTGTATTCAAT |
| | GACATTTTGTCCATTTCTCT | CTACCCTCTATTTCTAGGACAA |
| | CTATACCACCAACTCTCAT | CATGTTTTAATAATTCACCATA |
| | CTTGGTTACATTGCCTGTC | AATTAGTTCTATGAGCATTTTG |
| | AGTTGAACAGGCAAGATCT | ATCTAGTGGTTGTTGTAGATCA |
| | CCATTCCCAAAACACCACA | CAGGATATGAATAGATTGTGTG |
| | CTTATCTTGAATTGAGCCA | TATGTGTAAACCAGGAGTGTCT |
| | TCAAGAGGTATTTCTCTGA | TGACAAAAGGACACAGTGCTG |
| | TACACCATTATCTAAAACC | GTAAGACCAGCAGAAGAGGGT |
| | CAGTGTTATTTTTCAGTGA | CCCCTGTGGCAAAAATGTTTTA |
| | GGGTAGATTCCCTTTACTG | CTGACCTTTG (SEQ ID NO: 13) |
| | CTTTTTCTCCTCATTTCCA | |
| | (SEQ ID NO: 12) | |
| 7b | TGAATGTGGAAAGTGAAG | TCTATTGTCTCATAAGTCCTAC |
| | ATAACTACTAGTGCAGCAA | CACTCAGGAATAAGGCTTATAA |
| | GCAGACATAGAGATTTTAT | GAAACATGAGAAATGTGTAAC |
| | AAAAGTGGATGGTGAGCA | TGTAGAGTGAGTTAGACATAGC |
| | TTGTTTGTTAGATACAAAC | CAGCAGCTATATACGTACCCAT |
| | CATTAAGCAGAGATGCCTA | ATGCTCACAGTCTCTCTCCTTC |
| | GTGACTCAGAGTACAGAG | GCAGGATTAGGAAATAAAATT |
| | CTTCCTGACTGTCAACACT | GGGTGAAAAAGGTTATAGATA |
| | GACCATGACACTGACCACG | AGACAGAGATAGAAAGATTGC |
| | TTATGATCCAGGAATAATA | TAATCATTTAACATATTTGATT |
| | GAACCTGTATTTACTCTTA | TGGGCAAAGTAATTGAATACA |
| | GATATTCTTAAATTGTAAT | CTGTCAAGTAAAG (SEQ ID NO: |
| | CACAGAAGAGGACAAACA | 22) |
| | GTGATAG (SEQ ID NO: 21) | |
| 14a | GGTCTGTGCAAACAGTGTT | ACATCACCAGGGCTGGAGGTT |
| | TCTCATGCAACTTGGCTGC | ACATCCTGCTGGCCAAAACTAC |
| | CTTTAAATGGCTTACCAAC | ATTATGGTGGGTTAGCTAATTT |
| | TCTTTTCTGAAAAACCTAA | TACAGGAGGGATAAATTGTGA |
| | AAAATGTCTGTGTGCCAAG | GAATCTGGAAACCATAATCTTG |
| | TAAGATACTTCAATTCAAA | ACAAGAAAAAATTAACACCCA |
| | AGGAGGTTTTTCAATTTTT | ATTCCTTGGTGAGACTGGGCAT |
| | TCTCTCAGTTTATCATTTCT | TATATGGACATGGGAAATCTCA |
| | TCTACTTGAAAAATATATT | CAGTCATGATATATGTTAGGAA |
| | TTAATTTTAATGTTTTTTGT | GGAACTCTCCTTCAAGGGTCCA |
| | TTTATACAAAAATATATGA | GGACTGTAAGTATTGGCCTGCC |
| | ACTATGTATTACTATTGTC | CCAACACTTGG (SEQ ID NO: 17) |
| | ACCTGTCATTACCAAGAAG | |
| | T (SEQ ID NO: 16) | |
| 8a | CAAGAACATTAAAAAGAA | TATTTGTCAGAAAAATCTATCT |
| | ACAGGATCTCTCTTGCTTT | GCATACTCATTTCTTTGAATGA |
| | TGTTAAGAAAAAAAAACA | GATTACAATCATGAGATGTCCA |
| | GCAGCAGTACACCAAGAG | CTATCTGCATTTTTGATCACTA |
| | CAGTTAAATTACTCTATTT | GTGAAAACTCCTTCTTTCCAAA |
| | AGAGATGTCTGAAACACTT | GCCACTGGTGATTATTGTTTAT |
| | CTAAAGCAAAGCAGGTAA | TTAGGAGAGCAGGCCTTTAAA |
| | TACTTCAGGGACCATCTAC | AGAAATACTGTGGTCACCTGTG |
| | AAGCTTGCAGTTTTAGCTC | TGACTGAAAAAAGCACATATTC |
| | CCTTCTATAACTTTTCCAAT | AATTCATGTGAATAACATTAGA |
| | TTGATCTTTCTGTGGAAAA | AAGTTTCCAGGAACTGCTGATG |
| | ATACAAAATTGTGTCTGTT | ATTCAAGCA (SEQ ID NO: 24) |
| | TCTTGAAGACCGGTAACCA | |
| | GAAAGT (SEQ ID NO: 23) | |
| 13a | GAAACATCATTTATAGACT | TGCTTTGCTTGCACATGCAGTA |
| | AAGTGTGTATATTATAAAT | AACTTTTTTAATCTTAATTCATA |
| | CTGCTGATTGCATAGGTAA | AACTCTCACACTTTTACATTTC |
| | ATTTTTTTCAGTATCATCTG | CAATTTTCACATCTTTAATCAT |
| | CTGTCAAAATTTTGGCTGT | CTGGGAGATGATTTCACATCCT |
| | GATGAAATCAACTTTCTTC | CATCAAGTTTGTGGATGACCCA |
| | ATAGAAACTCATATAATGC | GAACTGAGGGCAGTGGCTAAC |
| | TGTGCTTCGGATTTTTGAT | TCACCAGAGTGCTGTGCTGCCA |
| | GAAAATAGTGATGATAAT | TCTGGAGGAACTTGCACAGTTG |
| | ACACCAGTGTTCCAGTTGT | GAGAAGGGCTGACAGGAACTT |
| | GACAGAGCAGTGCTTACA | GATGGAGTTCTACATGGAGAA |
| | GAGTCAAAAACTTTTTATT | GGGAAAGTTC (SEQ ID NO: 26) |
| | TTATTGTGCTATCCTGCCA | |
| | ATGA (SEQ ID NO: 25) | |

Insertion of the Plasmids into Male-Derived Lohmann-LSL PGC

To facilitate the integration of the DsRed gene into the desired SHL on the Z chromosome, PGC were co-transfected with CRISPR/Cas9 and DsRed HDR plasmids.

The CRISPR/Cas9 plasmid induces a double-strand break at the targeted genomic locus, while the DsRed plasmid facilitates repair by integrating the DsRed cassette at the cleaved site.

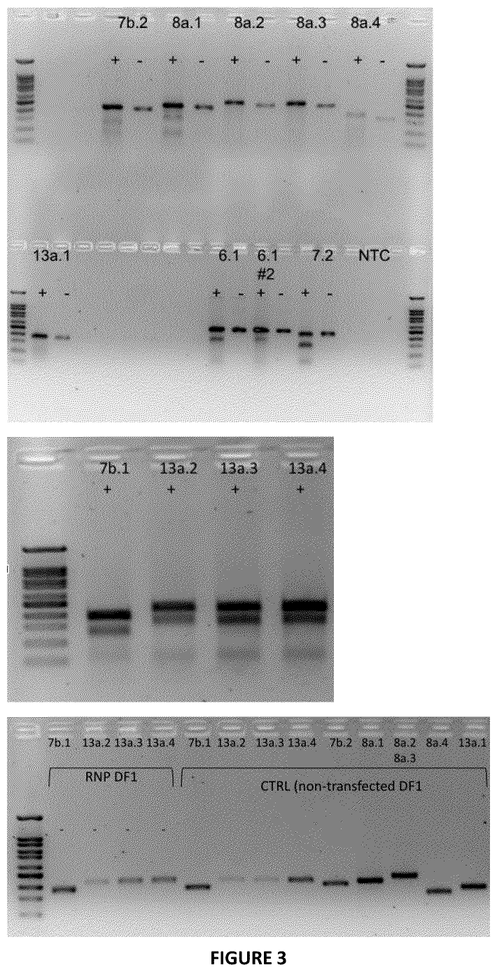

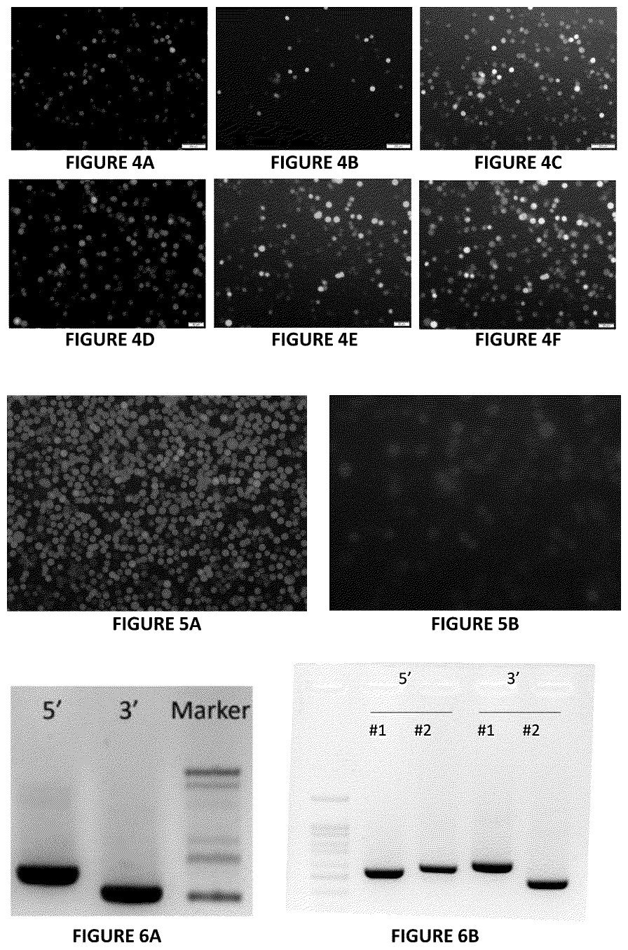

Two to three (2-3) days post-transfection, GFP and DsRed fluorescent signals were observed in the PGCs (FIG. 4). These signals displayed distinct green and red fluorescence, with numerous cells being positive for both signals.

### Sorting Procedures for the Enrichment of the DsRed-Positive Cells

To isolate cells exhibiting stable expression of the DsRed fluorescent protein, the red cell population underwent multiple rounds of sorting procedures. For each specific genomic region targeted, cells were sorted approximately 4 times, every 1-2 weeks, until a consistently stable red PGC culture was achieved (FIG. 5).

Among the five targeted SHL, three demonstrated robust and consistent fluorescence within their cultures, alongside sustained proliferation: 4a, 14a, and 13a. These three stable DsRed⁺ PGC cultures were subsequently expanded.

Molecular Validation of the Correct DsRed Integration into the Desired SHL

To validate the accurate integration of the DsRed cassette within the SHL on the Z chromosome of the three stable PGC cultures, two PCR assays were conducted with primers flanking either the 5′ or the 3′ homology arms of the cassette (Table 10).

TABLE 10
PCR primers for validating integration of DsRed cassette in a SHL

| SHL | Region | Forward Primer | Reverse Primer |
|---|---|---|---|
| 4a | 5′ | CACAGGGAGATTTGACAG | GGGCGTACTTGGCATAT |
| | | TGG (SEQ ID NO: 27) | GAT (SEQ ID NO: 28) |
| | 3′ | CCCCCTGAACCTGAAACA | GAGACTTTTGTAGCTTGA |
| | | TA (SEQ ID NO: 29) | CTTGG (SEQ ID NO: 30) |
| 13a (Set #1) | 5′ | CAGATCTTGGTTGAGATG | AAGTGGGCAGTTTACCG |
| | | CATC (SEQ ID NO: 31) | TAAATAC (SEQ ID NO: 32) |
| | 3′ | CCCCCTGAACCTGAAACA | GTTCTAAGGGAAGGACC |
| | | TA (SEQ ID NO: 33) | ACGT (SEQ ID NO: 34) |
| 13a (Set #2) | 5′ | CAGATCTTGGTTGAGATG | GGGCGTACTTGGCATAT |
| | | CATC (SEQ ID NO: 35) | GAT (SEQ ID NO: 36) |
| | 3′ | GCATTCTAGTTGTGGTTTG | GTTCTAAGGGAAGGACC |
| | | TCC (SEQ ID NO: 37) | ACGT (SEQ ID NO: 38) |

The PCR analyses yielded the expected product sizes (FIG. 6), affirming successful amplification of the targeted sequences. Sanger sequencing further validated the expected sequence integration within the SHL.

### Example 3

### In-Ovo Injection of PGC to Validate their Fluorescent Characteristics

To demonstrate that genome integrated DsRed cells (e.g., DsRed-positive PGC) as disclosed herein are detectable through the shell of an egg, infertile eggs were injected with either control (e.g., non-edited PGCs) or gene-edited PGCs containing the DsRed gene. The number of injected cells was equivalent to the number of cells in an embryo of an egg in an embryonic day-3.

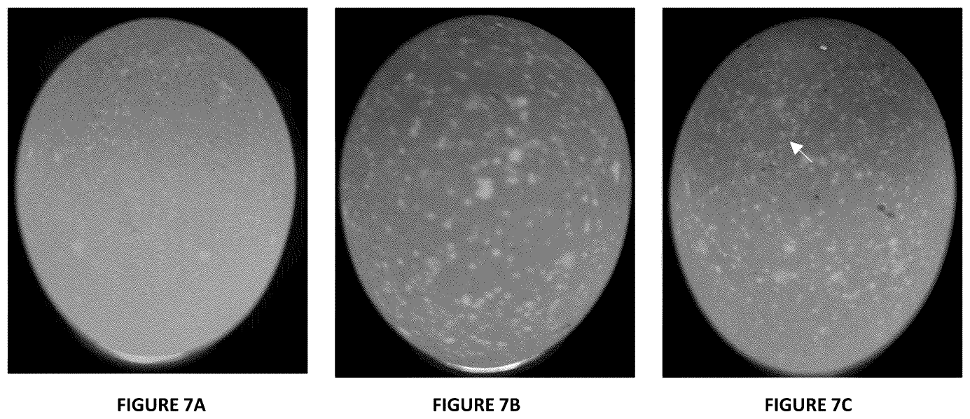

The results show that no fluorescent signal was detected by injection of control non-edited parental PGC (FIG. 7B). In sharp contrast, a distinct fluorescent focus was observed upon injection of DsRed-positive chicken PGCs.

Thus, it is concluded that chicken PGCs comprising DsRed encoding sequence integrated into a safe harbor site in the genome of the PGCs were successfully produced. A person of ordinary skill in the art would acknowledge that fully grown chicken are obtainable from the disclosed PGCs according to methods known in the art.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims which follow.

1. A transgenic female chicken comprising a recombinant Z gender chromosome comprising a nucleic acid sequence of an exogenous reporter gene encoding a red fluorescent protein (RFP), in at least one location of a Z gender chromosome of a female chicken, wherein said at least one location is listed under Table 1.

2. The transgenic female chicken of claim 1, wherein said at least one location is selected from the group consisting of: site 3, site 4, site 5, site 7, site 8, site 13, site 14, site 15, and any combination thereof.

3. The transgenic female chicken of claim 1, wherein said at least one location is listed under Table 3.

4. The transgenic female chicken of claim 3, wherein said at least one location is selected from the group consisting of: site 4a, site 5a, site 7b, site 13a, site 14a, and any combination thereof.

5. The transgenic female chicken of claim 4, wherein said at least one location is selected from the group consisting of: said site 4a, said site 13a, said site 14a, and any combination thereof.

6. The transgenic female chicken of claim 1, wherein said RFP is characterized by an excitation wavelength of 500-650 nm and an emission wavelength of 550-650 nm.

7. A cell obtained or derived from the transgenic female chicken of claim 1, and optionally wherein said cell is primordial germ cell (PGC).

8. (canceled)

9. A method of gender determination of a chicken embryo in an unhatched egg comprising the embryo within a structurally integral shell, the method comprising:
(a) obtaining at least one unhatched egg comprising an embryo within a structurally integral shell, from the transgenic female chicken of claim 1; and
(b) determining whether a red fluorescent signal is detected in said embryo residing in said unhatched egg, wherein detection of said red fluorescent signal indicates the expression of said RFP in said embryo within said structurally integral shell of said unhatched egg, and thus indicates the presence of said recombinant Z chromosome in said embryo,
thereby determining that said chicken embryo in said unhatched egg is a male embryo.

10. The method of claim 9, wherein detection of no red fluorescent signal in said embryo residing in said unhatched egg indicates that RFP is not expressed in said embryo, and thus indicates the absence of said recombinant Z chromosome in said embryo, thereby determining that said chicken embryo in said unhatched egg is a female embryo.

11. The method of claim 9, further comprising a step comprising subjecting said unhatched egg comprising the embryo within a structurally integral shell to a light source.

12. The method of claim 9, further comprising a step proceeding said step (b) comprising isolating at least one female embryo of said transgenic female chicken.

13. A kit comprising at least one first nucleic acid molecule encoding a guide RNA (gRNA), comprising the nucleotide sequence as set forth in any one of SEQ ID Nos: 1-3, and 18-20.

14. The kit of claim 13, further comprising at least one second nucleic acid molecule encoding any one of: a clustered regularly interspaced short palindromic repeat (CRISPR)-associated protein 9 (Cas9), an RFP, and both.

15. The kit of claim 14, further comprising instructions for integrating said at least second nucleic acid molecule encoding RFP into at least one location of Z gender chromosome of a female chicken, wherein said at least one location is listed under Table 1.

16. A method for producing a chicken comprising a recombinant Z gender chromosome, the method comprising:
(a) obtaining at least one transgenic PGC comprising a recombinant Z chromosome comprising a nucleic acid sequence of an exogenous reporter gene encoding an RFP in at least one location as listed under Table 1 in Z gender chromosome of a chicken; and,
(b) transplanting said transgenic PGC into a receptive chicken embryo,
thereby producing a chicken comprising the recombinant Z gender chromosome.

17. The method of claim 16, further comprising a step preceding step (a), comprising integrating said nucleic acid sequence of an exogenous reporter gene encoding an RFP into at least one location as listed under Table 1 in Z gender chromosome of a PGC, thereby obtaining said transgenic PGC.

18. The method of claim 16, wherein said at least one location is listed under Table 3.

19. The method of claim 16, wherein said at least one location is selected from the group consisting of: site 4a, site 13a, site 14a, and any combination thereof.

20. The method of claim 16, wherein said nucleic acid sequence of an exogenous reporter gene encoding an RFP is integrated into said location of said Z gender chromosome using CRISPR type II system, comprising Cas9 protein and a gRNA.

21. The method of claim 20, wherein said gRNA comprises the nucleotide sequence set forth in any one of SEQ ID Nos: 1, 3, and 20, and optionally wherein said nucleic acid sequence of an exogenous reporter gene is integrated into said site 4a, said site 13a, or said site 14a, using said Cas9 protein and a gRNA comprising the nucleotide sequence as set forth in SEQ ID NO: 1, SEQ ID NO: 20, or SEQ ID NO: 3, respectively.

22. (canceled)


SEQUENCE LISTING (41 sequences)

SEQ ID NO: 1 - RNA, 20 nt, Synthetic construct
  source 1..20: mol_type = other RNA
acatgcaatacactgaactg

SEQ ID NO: 2 - RNA, 20 nt, Synthetic construct
  source 1..20: mol_type = other RNA
actgttaacaaggttggttg

SEQ ID NO: 3 - RNA, 20 nt, Synthetic construct
  source 1..20: mol_type = other RNA
cttgtagggcttgattactg

SEQ ID NO: 4 - DNA, 2241 bp, Synthetic construct
  source 1..2241: mol_type = other DNA
ggcctatacagacaaatctttactaaatggagaatcctgacattttgtccatttctctctataccaccaactctcatcttggttacattgcctgtcagttgaacaggcaagatctccattcccaaaacaccacacttatcttgaattgagccatcaagaggtatttctctgatacaccattatctaaaacccagtgttatttttcagtgagggtagattccctttactgctttttctcctcatttccagaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatccataacggcattcagtacttgtcatcactaattagtgtattcaatctaccctctatttctaggacaacatgttttaataattcaccataaattagttctatgagcattttgatctagtggttgttgtagatcacaggatatgaatagattgtgtgtatgtgtaaaccaggagtgtcttgacaaaaggacacagtgctggtaagaccagcagaagagggtcccctgtggcaaaaatgttttactgacctttg

SEQ ID NO: 5 - DNA, 2243 bp, Synthetic construct
  source 1..2243: mol_type = other DNA
aatacacagcactgtatcttttatggcaatctattttcgattgtctcatgtgcagaacagttaccaggattgcaacaacggattgaatttatccgaaaagaagttttgtgtcctgctttgtgatagctgagaaagaaaggcagtgatgcttaaaaagcagtcagtgacctaatcacctactgtcaggtgtactatgaatacatacagtagagccagtaacacagtttgacagcattttcattagatgtttgaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatcccagaacaaaatagtatttttgttcacaactgggagtgaaatctgatttcaaaccactaaaaagaatagtggagacatgaagaaaaaacgttttgtctgaatgctttcttgggtagtcagaaataaaagctgttgtacggaagatcatatgaggctgctatgggtagcagcatcaagtgtggcagtggagcagagagagattgcatgccacggggagaggaaatgtggaaaattacacatatcaccgtgag

SEQ ID NO: 6 - DNA, 2243 bp, Synthetic construct
  source 1..2243: mol_type = other DNA
ggtctgtgcaaacagtgtttctcatgcaacttggctgcctttaaatggcttaccaactcttttctgaaaaacctaaaaaatgtctgtgtgccaagtaagatacttcaattcaaaaggaggtttttcaattttttctctcagtttatcatttcttctacttgaaaaatatattttaattttaatgttttttgttttatacaaaaatatatgaactatgtattactattgtcacctgtcattaccaagaagtgaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatccacatcaccagggctggaggttacatcctgctggccaaaactacattatggtgggttagctaattttacaggagggataaattgtgagaatctggaaaccataatcttgacaagaaaaaattaacacccaattccttggtgagactgggcattatatggacatgggaaatctcacagtcatgatatatgttaggaaggaactctccttcaagggtccaggactgtaagtattggcctgccccaacacttgg

SEQ ID NO: 7 - protein, 225 aa, Synthetic construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGSFIYKVKFIGVNFPSDGPVMQKKTMGWEASTERLYPRDGVLKGEIHKALKLKDGGHYLVEFKSIYMAKKPVQLPGYYYVDSKLDITSHNEDYTIVEQYERAEGRHHLFL

SEQ ID NO: 8 - DNA, 678 bp, Synthetic construct
  source 1..678: mol_type = other DNA
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtag

SEQ ID NO: 9 - DNA, 4203 bp, Synthetic construct
  source 1..4203: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagtaa

SEQ ID NO: 10 - DNA, 811 bp, Synthetic construct
  source 1..811: mol_type = other DNA
gaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccacc

SEQ ID NO: 11 - DNA, 254 bp, Synthetic construct
  source 1..254: mol_type = other DNA
cggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatcc

SEQ ID NO: 12 - DNA, 248 bp, Gallus gallus
  source 1..248: mol_type = other DNA
ggcctatacagacaaatctttactaaatggagaatcctgacattttgtccatttctctctataccaccaactctcatcttggttacattgcctgtcagttgaacaggcaagatctccattcccaaaacaccacacttatcttgaattgagccatcaagaggtatttctctgatacaccattatctaaaacccagtgttatttttcagtgagggtagattccctttactgctttttctcctcatttcca

SEQ ID NO: 13 - DNA, 250 bp, Gallus gallus
  source 1..250: mol_type = other DNA
ataacggcattcagtacttgtcatcactaattagtgtattcaatctaccctctatttctaggacaacatgttttaataattcaccataaattagttctatgagcattttgatctagtggttgttgtagatcacaggatatgaatagattgtgtgtatgtgtaaaccaggagtgtcttgacaaaaggacacagtgctggtaagaccagcagaagagggtcccctgtggcaaaaatgttttactgacctttg

SEQ ID NO: 14 - DNA, 250 bp, Gallus gallus
  source 1..250: mol_type = other DNA
aatacacagcactgtatcttttatggcaatctattttcgattgtctcatgtgcagaacagttaccaggattgcaacaacggattgaatttatccgaaaagaagttttgtgtcctgctttgtgatagctgagaaagaaaggcagtgatgcttaaaaagcagtcagtgacctaatcacctactgtcaggtgtactatgaatacatacagtagagccagtaacacagtttgacagcattttcattagatgttt

SEQ ID NO: 15 - DNA, 250 bp, Gallus gallus
  source 1..250: mol_type = other DNA
cagaacaaaatagtatttttgttcacaactgggagtgaaatctgatttcaaaccactaaaaagaatagtggagacatgaagaaaaaacgttttgtctgaatgctttcttgggtagtcagaaataaaagctgttgtacggaagatcatatgaggctgctatgggtagcagcatcaagtgtggcagtggagcagagagagattgcatgccacggggagaggaaatgtggaaaattacacatatcaccgtgag

SEQ ID NO: 16 - DNA, 250 bp, Gallus gallus
  source 1..250: mol_type = other DNA
ggtctgtgcaaacagtgtttctcatgcaacttggctgcctttaaatggcttaccaactcttttctgaaaaacctaaaaaatgtctgtgtgccaagtaagatacttcaattcaaaaggaggtttttcaattttttctctcagtttatcatttcttctacttgaaaaatatattttaattttaatgttttttgttttatacaaaaatatatgaactatgtattactattgtcacctgtcattaccaagaagt

SEQ ID NO: 17 - DNA, 250 bp, Gallus gallus
  source 1..250: mol_type = other DNA
acatcaccagggctggaggttacatcctgctggccaaaactacattatggtgggttagctaattttacaggagggataaattgtgagaatctggaaaccataatcttgacaagaaaaaattaacacccaattccttggtgagactgggcattatatggacatgggaaatctcacagtcatgatatatgttaggaaggaactctccttcaagggtccaggactgtaagtattggcctgccccaacacttgg

SEQ ID NO: 18 - RNA, 20 nt, Synthetic construct
  source 1..20: mol_type = other RNA
tcttgctttccactttccat

SEQ ID NO: 19 - RNA, 20 nt, Synthetic construct
  source 1..20: mol_type = other RNA
aagtgtggaacaaactgctg

SEQ ID NO: 20 - RNA, 20 nt, Synthetic construct
  source 1..20: mol_type = other RNA
gatgggctccacaaggaact

SEQ ID NO: 21 - DNA, 250 bp, Gallus gallus
  source 1..250: mol_type = other DNA
tgaatgtggaaagtgaagataactactagtgcagcaagcagacatagagattttataaaagtggatggtgagcattgtttgttagatacaaaccattaagcagagatgcctagtgactcagagtacagagcttcctgactgtcaacactgaccatgacactgaccacgttatgatccaggaataatagaacctgtatttactcttagatattcttaaattgtaatcacagaagaggacaaacagtgatag

SEQ ID NO: 22 - DNA, 250 bp, Gallus gallus
  source 1..250: mol_type = other DNA
tctattgtctcataagtcctaccactcaggaataaggcttataagaaacatgagaaatgtgtaactgtagagtgagttagacatagccagcagctatatacgtacccatatgctcacagtctctctccttcgcaggattaggaaataaaattgggtgaaaaaggttatagataagacagagatagaaagattgctaatcatttaacatatttgatttgggcaaagtaattgaatacactgtcaagtaaag

SEQ ID NO: 23 - DNA, 250 bp, Gallus gallus
  source 1..250: mol_type = other DNA
caagaacattaaaaagaaacaggatctctcttgcttttgttaagaaaaaaaaacagcagcagtacaccaagagcagttaaattactctatttagagatgtctgaaacacttctaaagcaaagcaggtaatacttcagggaccatctacaagcttgcagttttagctcccttctataacttttccaatttgatctttctgtggaaaaatacaaaattgtgtctgtttcttgaagaccggtaaccagaaagt

SEQ ID NO: 24 - DNA, 250 bp, Gallus gallus
  source 1..250: mol_type = other DNA
tatttgtcagaaaaatctatctgcatactcatttctttgaatgagattacaatcatgagatgtccactatctgcatttttgatcactagtgaaaactccttctttccaaagccactggtgattattgtttatttaggagagcaggcctttaaaagaaatactgtggtcacctgtgtgactgaaaaaagcacatattcaattcatgtgaataacattagaaagtttccaggaactgctgatgattcaagca

SEQ ID NO: 25 - DNA, 250 bp, Gallus gallus
  source 1..250: mol_type = other DNA
gaaacatcatttatagactaagtgtgtatattataaatctgctgattgcataggtaaatttttttcagtatcatctgctgtcaaaattttggctgtgatgaaatcaactttcttcatagaaactcatataatgctgtgcttcggatttttgatgaaaatagtgatgataatacaccagtgttccagttgtgacagagcagtgcttacagagtcaaaaactttttattttattgtgctatcctgccaatga

SEQ ID NO: 26 - DNA, 250 bp, Gallus gallus
  source 1..250: mol_type = other DNA
tgctttgcttgcacatgcagtaaacttttttaatcttaattcataaactctcacacttttacatttccaattttcacatctttaatcatctgggagatgatttcacatcctcatcaagtttgtggatgacccagaactgagggcagtggctaactcaccagagtgctgtgctgccatctggaggaacttgcacagttggagaagggctgacaggaacttgatggagttctacatggagaagggaaagttc

SEQ ID NO: 27 - DNA, 21 bp, Synthetic construct
  source 1..21: mol_type = other DNA
cacagggagatttgacagtgg

SEQ ID NO: 28 - DNA, 20 bp, Synthetic construct
  source 1..20: mol_type = other DNA
gggcgtacttggcatatgat

SEQ ID NO: 29 - DNA, 20 bp, Synthetic construct
  source 1..20: mol_type = other DNA
ccccctgaacctgaaacata

SEQ ID NO: 30 - DNA, 23 bp, Synthetic construct
  source 1..23: mol_type = other DNA
gagacttttgtagcttgacttgg

SEQ ID NO: 31 - DNA, 22 bp, Synthetic construct
  source 1..22: mol_type = other DNA
cagatcttggttgagatgcatc

SEQ ID NO: 32 - DNA, 24 bp, Synthetic construct
  source 1..24: mol_type = other DNA
aagtgggcagtttaccgtaaatac

SEQ ID NO: 33 - DNA, 20 bp, Synthetic construct
  source 1..20: mol_type = other DNA
ccccctgaacctgaaacata

SEQ ID NO: 34 - DNA, 21 bp, Synthetic construct
  source 1..21: mol_type = other DNA
gttctaagggaaggaccacgt

SEQ ID NO: 35 - DNA, 22 bp, Synthetic construct
  source 1..22: mol_type = other DNA
cagatcttggttgagatgcatc

SEQ ID NO: 36 - DNA, 20 bp, Synthetic construct
  source 1..20: mol_type = other DNA
gggcgtacttggcatatgat

SEQ ID NO: 37 - DNA, 22 bp, Synthetic construct
  source 1..22: mol_type = other DNA
gcattctagttgtggtttgtcc

SEQ ID NO: 38 - DNA, 21 bp, Synthetic construct
  source 1..21: mol_type = other DNA
gttctaagggaaggaccacgt

SEQ ID NO: 39 - DNA, 2243 bp, Synthetic construct
  source 1..2243: mol_type = other DNA
tgaatgtggaaagtgaagataactactagtgcagcaagcagacatagagattttataaaagtggatggtgagcattgtttgttagatacaaaccattaagcagagatgcctagtgactcagagtacagagcttcctgactgtcaacactgaccatgacactgaccacgttatgatccaggaataatagaacctgtatttactcttagatattcttaaattgtaatcacagaagaggacaaacagtgataggaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatcctctattgtctcataagtcctaccactcaggaataaggcttataagaaacatgagaaatgtgtaactgtagagtgagttagacatagccagcagctatatacgtacccatatgctcacagtctctctccttcgcaggattaggaaataaaattgggtgaaaaaggttatagataagacagagatagaaagattgctaatcatttaacatatttgatttgggcaaagtaattgaatacactgtcaagtaaag

SEQ ID NO: 40 - DNA, 2243 bp, Synthetic construct
  source 1..2243: mol_type = other DNA
caagaacattaaaaagaaacaggatctctcttgcttttgttaagaaaaaaaaacagcagcagtacaccaagagcagttaaattactctatttagagatgtctgaaacacttctaaagcaaagcaggtaatacttcagggaccatctacaagcttgcagttttagctcccttctataacttttccaatttgatctttctgtggaaaaatacaaaattgtgtctgtttcttgaagaccggtaaccagaaagtgaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatcctatttgtcagaaaaatctatctgcatactcatttctttgaatgagattacaatcatgagatgtccactatctgcatttttgatcactagtgaaaactccttctttccaaagccactggtgattattgtttatttaggagagcaggcctttaaaagaaatactgtggtcacctgtgtgactgaaaaaagcacatattcaattcatgtgaataacattagaaagtttccaggaactgctgatgattcaagca

SEQ ID NO: 41 - DNA, 2243 bp, Synthetic construct
  source 1..2243: mol_type = other DNA
gaaacatcatttatagactaagtgtgtatattataaatctgctgattgcataggtaaatttttttcagtatcatctgctgtcaaaattttggctgtgatgaaatcaactttcttcatagaaactcatataatgctgtgcttcggatttttgatgaaaatagtgatgataatacaccagtgttccagttgtgacagagcagtgcttacagagtcaaaaactttttattttattgtgctatcctgccaatgagaattccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggcgccaccatggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctccttcatctacaaggtgaagttcatcggcgtgaacttcccctccgacggccccgtaatgcagaagaagactatgggctgggaggcctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcgccgagggccgccaccacctgttcctgtagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtggatcctgctttgcttgcacatgcagtaaacttttttaatcttaattcataaactctcacacttttacatttccaattttcacatctttaatcatctgggagatgatttcacatcctcatcaagtttgtggatgacccagaactgagggcagtggctaactcaccagagtgctgtgctgccatctggaggaacttgcacagttggagaagggctgacaggaacttgatggagttctacatggagaagggaaagttc